(19) United States
(12) Patent Application Publication
(10) Pub. No.: US 2006/0240065 A1
(43) Pub. Date: Oct. 26, 2006

(54) Compositions for medical devices containing agent combinations in controlled volumes

(76) Inventor: Yung-Ming Chen, Cupertino, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/115,631

(22) Filed: Apr. 26, 2005

(51) Int. Cl. A61F 2/00; A61K 9/50; A61K 9/16; A61K 31/4745

(52) U.S. Cl. 424/423; 424/490; 977/906; 514/291

(57) ABSTRACT

The present invention generally encompasses controlled-volume materials that may, for example, be in a medical device or applied on a medical device as a coating, as well as methods of applying these materials.

### BACKGROUND

1. Field of the Invention

This invention generally relates to medical devices and, more particularly, medical devices containing a combination of agents.

2. Description of the State of the Art

A current paradigm in biomaterials research is the control of protein adsorption on an implant surface. Uncontrolled protein adsorption on an implant surface is a problem with current biomaterial implants and leads to a mixed layer of partially denatured proteins on the implant surface. This mixed layer of partially denatured proteins leads to disease, for example, by providing cell-binding sites from adsorbed plasma proteins such as fibrinogen and immunoglobulin G. Platelets and inflammatory cells such as, for example, monocytes, macrophages and neutrophils, adhere to the cell-binding sites. A wide variety of proinflammatory and proliferative factors may be secreted and result in a diseased state. Accordingly, a non-fouling surface, which is a surface that does not become fouled or becomes less fouled with this layer of partially denatured proteins, is desirable.

A stent is an example of an implant that can benefit from a non-fouling surface. Stents are a mechanical intervention that can be used as a vehicle for delivering pharmaceutically active agents. As a mechanical intervention, stents can physically hold open and, if desired, expand a passageway within a subject. Typically, a stent may be compressed, inserted into a small vessel through a catheter, and then expanded to a larger diameter once placed in a proper location. Examples of patents disclosing stents include U.S. Pat. Nos. 4,733,665, 4,800,882 and 4,886,062.

Stents play an important role in a variety of medical procedures such as, for example, percutaneous transluminal coronary angioplasty (PTCA), which is a procedure used to treat heart disease. In PTCA, a balloon catheter is inserted through a brachial or femoral artery, positioned across a coronary artery occlusion, inflated to compress atherosclerotic plaque and open the lumen of the coronary artery, deflated and withdrawn. Problems with PTCA include formation of intimal flaps or torn arterial linings, both of which can create another occlusion in the lumen of the coronary artery. Moreover, thrombosis and restenosis may occur several months after the procedure and create a need for additional angioplasty or a surgical by-pass operation. Stents are generally implanted to reduce occlusions, inhibit thrombosis and restenosis, and maintain patency within vascular lumens such as, for example, the lumen of a coronary artery.

Stents are also being developed to provide for local delivery of agents. Local delivery of agents is often preferred over systemic delivery of agents, particularly where high systemic doses are necessary to achieve an effect at a particular site within a subject—high systemic doses of agents can often create adverse effects within the subject. One proposed method of local delivery includes coating the surface of a medical article with a polymeric carrier and attaching an agent to, or blending it with, the polymeric carrier.

Agent-coated stents have demonstrated dramatic reductions in the rates of stent restenosis by inhibiting tissue growth associated with the restenosis. Restenosis is a very complicated process and agents have been applied in combination in an attempt to circumvent the process of restenosis. One method of applying multiple agents involves blending the agents together in one formulation and applying the blend to the surface of a stent in a polymer matrix. A disadvantage of this method is that the agents are released from the matrix through the blend and compete with one another for release. Control over the release of agents is an important design consideration and the next hallmark in the development of stent technology.

The release profile of the agents from such a matrix is difficult to control. In some applications, control over the release profile of the agents can be important to providing the effects sought from the agents. There are numerous agent-release considerations in a polymer coating matrix including, but not limited to: functional groups variations on polymers in the matrix; the morphology of the polymers the matrix, which can be solute dependent; solubility parameters of the polymers in a matrix, which affects polymer compatibility and morphology. The manner in which the agents are combined with the polymers can also have a profound effect such as, for example, whether the agents are bonded or blended with the polymers. Interactions between the agents can also affect the release profile of the agents.

Accordingly, there is a need for medical devices and coatings that include a combination of agents, wherein each of the agents (i) can be incorporated in the device or coating without cross-contamination from the other agents; (ii) can perform its function substantially free from interference from the other agents, (ii) can be incorporated in the device or coating such that the agent has a predetermined release rate and absorption rate; and (iv) can be combined with other agents that are bioactive, biobeneficial, diagnostic, and/or control a physical property or a mechanical property of a medical device.

### SUMMARY

Embodiments of the present invention generally encompass controlled-volume materials that may, for example, be in a medical device or applied on a medical device as a coating. These materials may be used in medical devices that comprise stents. In some embodiments, the invention can include a medical device comprising a combination of agents, wherein an agent within the combination of agents is positioned within a controlled volume at one or more predetermined regions on a medical device, within the medical device, within a coating on the medical device, or a combination thereof.

In other embodiments, the invention can include a coating for a medical device comprising a combination of agents, wherein an agent is positioned within a controlled volume at one or more predetermined regions on the device, within the device, within a coating on the device, or a combination thereof. In other embodiments, the invention can include a method of coating a medical device comprising selecting a combination of agents; and applying an agent from the combination of agents within one or more controlled volumes at one or more predetermined regions on a medical device, within the device, within a coating for the device, or a combination thereof, such that the coating comprises the one or more controlled volumes.

In other embodiments, the invention can include a coating for a medical device comprising a combination of agents, wherein the coating is formed using a process comprising selecting a combination of agents. The combination of agents can include everolimus, clobetasol, tacrolimus, rapamycin, ABT-578, or any combination thereof. The process includes applying an agent from the combination of agents within one or more controlled volumes at one or more predetermined regions on a medical device, within the device, within a coating for the device, or a combination thereof, such that the coating comprises the one or more controlled volumes. The applying comprises forming the controlled volumes through a method that includes the use of acoustic energy.

### BRIEF DESCRIPTION OF THE DRAWINGS

FIG. 1 depicts an example of a three-dimensional view of a stent 101 according to some embodiments of the present invention.

FIG. 2 illustrates select areas of an abluminal portion of a stent that can be selectively coated with a combination of agents according to some embodiments of the present invention.

FIGS. 3a and 3b illustrate a sandwiched-coating design according to some embodiments of the present invention.

FIG. 4 illustrates a checkerboard-type coating design by showing a top view of the abluminal surface of a stent that was coated in sections according to some embodiments of the present invention.

FIGS. 5a and 5b illustrate an engraved-type coating design by showing a top view of the abluminal surface of a stent with engravings according to some embodiments of the present invention.

FIG. 6 illustrates a stent coating apparatus according to some embodiments of the present invention.

FIGS. 7a-7c illustrate an assembly that incorporates a nozzle according to some embodiments of the present invention.

FIGS. 8a and 8b illustrate an ejector assembly that does not require a nozzle according to some embodiments of the present invention.

FIG. 9 illustrates a method of ejecting the controlled-volumes downward onto the abluminal surface of a stent according to some embodiments of the present invention.

FIGS. 10a and 10b illustrate alternative designs of an acoustic ejector assembly according to some embodiments of the present invention.

### DETAILED DESCRIPTION

As discussed in more detail below, embodiments of the present invention generally encompass compositions that include a combination of agents such as, for example, therapeutic, prophylactic, diagnostic and/or other agents, for use with medical articles. The invention also encompasses methods for fabricating the compositions. The medical articles comprise any medical device such as, for example, an implantable medical device such as a stent. In some embodiments, the compositions can be used as a coating on the implantable substrate. In other embodiments, a medical device such as a stent is made in whole or in part from the composition.

An “agent” can be a moiety that may be bioactive, biobeneficial, diagnostic, plasticizing, or have a combination of these characteristics. A “moiety” can be a functional group composed of at least 1 atom, a bonded residue in a macromolecule, an individual unit in a copolymer or an entire polymeric block. It is to be appreciated that any medical devices that can be improved through the teachings described herein are within the scope of the present invention.

Examples of medical devices include, but are not limited to, stents, stent-grafts, vascular grafts, artificial heart valves, foramen ovale closure devices, cerebrospinal fluid shunts, pacemaker electrodes, guidewires, ventricular assist devices, cardiopulmonary bypass circuits, blood oxygenators, coronary shunts (AXIUS™, Guidant Corp.) and endocardial leads (FINELINE® and ENDOTAK®, Guidant Corp.). In some embodiments, the stents include, but are not limited to, tubular stents, self-expanding stents, coil stents, ring stents, multi-design stents, and the like. In other embodiments, the stents are metallic; low-ferromagnetic; non-ferromagnetic; biostable polymeric; biodegradable polymeric or biodegradable metallic. In some embodiments, the stents include, but are not limited to, vascular stents, renal stents, biliary stents, pulmonary stents and gastrointestinal stents

The medical devices can be comprised of a metal or an alloy, including, but not limited to, ELASTINITE® (Guidant Corp.), NITINOL® (Nitinol Devices and Components), stainless steel, tantalum, tantalum-based alloys, nickel-titanium alloy, platinum, platinum-based alloys such as, for example, platinum-iridium alloys, iridium, gold, magnesium, titanium, titanium-based alloys, zirconium-based alloys, alloys comprising cobalt and chromium (ELGILOY®, Elgiloy Specialty Metals, Inc.; MP35N and MP20N, SPS Technologies) or combinations thereof. The tradenames “MP35N” and “MP20N” describe alloys of cobalt, nickel, chromium and molybdenum. The MP35N consists of 35% cobalt, 35% nickel, 20% chromium, and 10% molybdenum. The MP20N consists of 50% cobalt, 20% nickel, 20% chromium, and 10% molybdenum. Medical devices with structural components that are comprised of bioabsorbable polymers or biostable polymers are also included within the scope of the present invention.

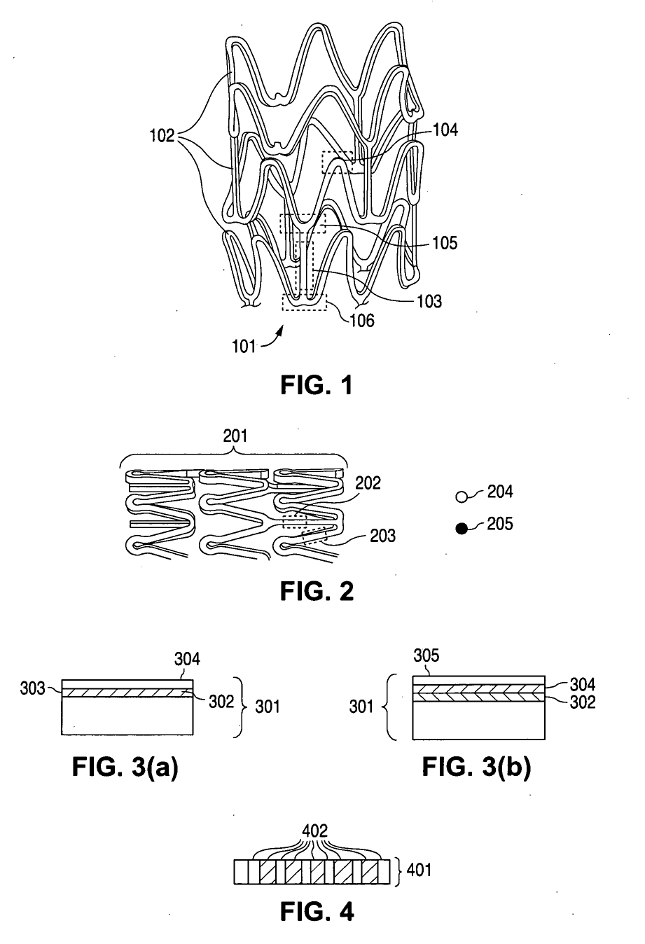

Embodiments of the devices described herein may be illustrated by a stent. FIG. 1 depicts an example of a three-dimensional view of a stent 101 according to some embodiments of the present invention. The stent may be made up of a pattern of a number of interconnecting structural elements or struts 102. The embodiments disclosed herein are not limited to stents or to the stent pattern illustrated in FIG. 1 and are easily applicable to other patterns and other devices. The variations in the structure of patterns are virtually unlimited.

Many medical implants undergo a great deal of strain during their manufacture and use that can result in structural failure. Structural failure can occur as a result of manipulating the implant in preparation for placing the implant in a subject and while placing the implant in a desired location in a subject. Typically, a stent may be compressed, inserted into a small vessel through a catheter, and then expanded to a larger diameter in a subject. In some embodiments, the agent-containing compositions can be applied in the form of a controlled volume, such as a droplet, in select areas of a stent where the struts 102 are subject to less stress and strain upon expansion and contraction of the stent. Application of the agents in low strain areas 103 of a stent, for example, can avoid problems, such as cracking and flaking, that can occur in high strain regions 104, 105, 106 of the stent.

In other embodiments, the agent-containing compositions can be applied selectively to an abluminal surface of a medical device such as, for example, a stent. In most embodiments, the stent can be an balloon-expandable stent or a self-expandable stent. The “abluminal” surface refers to the surface of the device that is directed away from the lumen of the organ in which the device has been deployed. In one example the lumen is an arterial lumen, and the abluminal surface of the stent is the surface that is placed in contact with the inner wall of the artery.

FIG. 2 illustrates select areas of an abluminal portion of a stent that can be selectively coated with a combination of agents using controlled-volume coating methods of the present invention. In this embodiment, agent A 204 can be selectively applied to area 202, and agent B 205 can be selectively applied to area 203. This selective application of agents allows for a controlled release of each agent by allowing for the independent selection of the manner in which each agent is attached to a surface of the stent 201. For example, an agent may be applied by itself, combined with a polymer that affects the rate of release of the agent, sandwiched between polymer layers, encapsulated within a polymer network, or any combination thereof.

The Agent-Containing Compositions

The agent-containing compositions of the present invention include any combination of polymers, copolymers and agents, wherein the combination comprises a combination of agents. The polymers comprising the combination of agents can be biodegradable, for example, due to the labile nature of chemical functionalities within the polymer network such as, for example, ester groups that can be present between chemical moieties. Accordingly, these compositions can be designed such that they can be broken down, absorbed, resorbed and eliminated by a mammal. The compositions of the present invention can be used, for example, to form medical articles and coatings.

The terms “combine,” “combined,” “combining,” and “combination” all refer to a relationship between components of a composition and include blends, mixtures, linkages, and combinations thereof, of components that form the compositions. The linkages can be connections that are physical, chemical, or a combination thereof. Examples of physical connections include, but are not limited to, an interlinking of components that can occur, for example, in interpenetrating networks and chain entanglement. Examples of chemical connections include, but are not limited to, covalent and non-covalent bonds. Covalent bonds include, but are not limited to, simple covalent bonds and coordinate bonds. Non-covalent bonds include, but are not limited to, ionic bonds, and inter-molecular attractions such as, for example, hydrogen bonds and attractions created by induced and permanent dipole-dipole interactions.

Compositions that are selected for an in vivo use should meet particular requirements with regard to physical, mechanical, chemical, and biological properties of the compositions. An example of a physical property that can affect the performance of a biodegradable composition in vivo is water uptake. An example of a mechanical property that can affect the performance of a composition in vivo is the ability of the composition to withstand stresses that can cause mechanical failure of the composition such as, for example, cracking, flaking, peeling, and fracturing. An example of a chemical property that can affect performance of a biodegradable composition in vivo is the rate of absorption of the composition by a subject. An example of a biological property that can affect performance of a composition in vivo is the bioactive and/or biobeneficial nature of the composition, both of which are described below. The terms “subject” and “patient” can be used interchangeably and refer to an animal such as a mammal including, but not limited to, non-primates such as, for example, a cow, pig, horse, cat, dog, rat, and mouse; and primates such as, for example, a monkey or a human.

While not intending to be bound by any theory or mechanism of action, water uptake by a composition can be an important characteristic in the design of a composition. Water can act as a plasticizer for modifying the mechanical properties of the composition. Control of water uptake can also provide some control over the hydrolysis of a coating and thus can provide control over the degradation rate, absorption rate, and the agent release rate of a medical article or coating in vivo. In some embodiments, an increase in hydrolysis can also increase the release rate of an agent by creating channels within a medical article or coating that can serve as transport pathways for diffusion of the agents from the composition within a subject.

The compositions of the present invention can be used to form medical devices and coatings that include a combination of agents, wherein each of the agents (i) can be incorporated in the device or coating without cross-contamination from the other agents; (ii) can perform its function substantially free from interference from the other agents, (ii) can be incorporated in the device or coating such that the agent has a predetermined release rate and absorption rate; and (iv) can be combined with other agents that are bioactive, biobeneficial, diagnostic, and/or control a physical property or a mechanical property of a medical device.

For the purposes of the present invention, a polymer or coating is “biodegradable” when it is capable of being completely or substantially degraded or eroded-when exposed to an in vivo environment or a representative in vitro. A polymer or coating is capable of being degraded or eroded when it can be gradually broken-down, resorbed, absorbed and/or eliminated by, for example, hydrolysis, enzymolysis, oxidation, metabolic processes, bulk or surface erosion, and the like within a subject. It should be appreciated that traces or residue of polymer may remain on the device, near the site of the device, or near the site of a biodegradable device, following biodegradation. The terms “bioabsorbable” and “biodegradable” are used interchangeably in this application.

The polymers used in the present invention may be biodegradable and may include, but are not limited to, condensation copolymers and should be chosen according to a desired performance parameter of a product that will be formed from the composition. Such performance parameters may include, for example, the toughness of a medical device or coating, the capacity for the loading concentration of an agent, and the rate of biodegradation and elimination of the composition from a subject. If the other polymers in a composition are non-biodegradable, they should be sized to produce polymer fragments that can clear from the subject following biodegradation of the composition.

In most embodiment, the polymers that can be used include natural or synthetic polymers; homopolymers and copolymers, such as, for example, copolymers that are random, alternating, block, graft, and/or crosslinked; or any combination and/or blend thereof. The copolymers include polymers with more than two different types of repeating units such as, for example, terpolymers.

In some embodiments, the number average molecular weight of the polymer fragments should be at or below about 40,000 Daltons, or any range therein. In other embodiments, the molecular weight of the fragments range from about 300 Daltons to about 40,000 Daltons, from about 8,000 Daltons to about 30,000 Daltons, from about 10,000 Daltons to about 20,000 Daltons, or any range therein. The molecular weights are taught herein as a number average molecular weight.

Examples of polymers that can be combined with the agents of the present invention include, but are not limited to, poly(acrylates) such as poly(butyl methacrylate), poly(ethyl methacrylate), poly(hydroxyl ethyl methacrylate), poly(ethyl methacrylate-co-butyl methacrylate), copolymers of ethylene-methyl methacrylate; poly (2-acrylamido-2-methylpropane sulfonic acid), and polymers and copolymers of aminopropyl methacrylamide; poly(cyanoacrylates); poly(carboxylic acids); poly(vinyl alcohols); poly(maleic anhydride) and copolymers of maleic anhydride; fluorinated polymers or copolymers such as poly(vinylidene fluoride), poly(vinylidene fluoride-co-hexafluoro propene), poly(tetrafluoroethylene), and expanded poly(tetrafluoroethylene); poly(sulfone); poly(N-vinyl pyrrolidone); poly(aminocarbonates); poly(iminocarbonates); poly(anhydride-co-imides), poly(hydroxyvalerate); poly(L-lactic acid); poly(L-lactide); poly(caprolactones); poly(lactide-co-glycolide); poly(hydroxybutyrates); poly(hydroxybutyrate-co-valerate); poly(dioxanones); poly(orthoesters); poly(anhydrides); poly(glycolic acid); poly(glycolide); poly(D,L-lactic acid); poly(D,L-lactide); poly(glycolic acid-co-trimethylene carbonate); poly(phosphoesters); poly(phosphoester urethane); poly(trimethylene carbonate); poly(iminocarbonate); poly(ethylene); poly(propylene) co-poly(ether-esters) such as, for example, poly(dioxanone) and poly(ethylene oxide)/poly(lactic acid); poly(anhydrides), poly(alkylene oxalates); poly(phosphazenes); poly(urethanes); silicones; poly(esters; poly(olefins); copolymers of poly(isobutylene); copolymers of ethylene-alphaolefin; vinyl halide polymers and copolymers such as poly(vinyl chloride); poly(vinyl ethers) such as poly(vinyl methyl ether); poly(vinylidene halides) such as, for example, poly(vinylidene chloride); poly(acrylonitrile); poly(vinyl ketones); poly(vinyl aromatics) such as poly(styrene); poly(vinyl esters) such as poly(vinyl acetate); copolymers of vinyl monomers and olefins such as poly(ethylene-co-vinyl alcohol) (EVAL), copolymers of acrylonitrile-styrene, ABS resins, and copolymers of ethylene-vinyl acetate; poly(amides) such as Nylon 66 and poly(caprolactam); alkyd resins; poly(carbonates); poly(oxymethylenes); poly(imides); poly(ester amides); poly(ethers) including poly(alkylene glycols) such as, for example, poly(ethylene glycol) and poly(propylene glycol); epoxy resins; polyurethanes; rayon; rayon-triacetate; biomolecules such as, for example, fibrin, fibrinogen, starch, poly(amino acids); peptides, proteins, gelatin, chondroitin sulfate, dermatan sulfate (a copolymer of D-glucuronic acid or L-iduronic acid and N-acetyl-D-galactosamine), collagen, hyaluronic acid, and glycosaminoglycans; other polysaccharides such as, for example, poly(N-acetylglucosamine), chitin, chitosan, cellulose, cellulose acetate, cellulose butyrate, cellulose acetate butyrate, cellophane, cellulose nitrate, cellulose propionate, cellulose ethers, and carboxymethylcellulose; and derivatives, analogs, homologues, congeners, salts, copolymers and combinations thereof. In some embodiments, the polymers are selected such that they specifically exclude any one or any combination of these polymers.

In some embodiments, the polymers can be biodegradable. Examples of biodegradable polymers include, but are not limited to, polymers having repeating units such as, for example, an α-hydroxycarboxylic acid, a cyclic diester of an α-hydroxycarboxylic acid, a dioxanone, a lactone, a cyclic carbonate, a cyclic oxalate, an epoxide, a glycol, an anhydride, a lactic acid, a glycolic acid, a lactide, a glycolide, an ethylene oxide, an ethylene glycol, or combinations thereof. In other embodiments, the biodegradable polymers include, but are not limited to, polyesters, poly(ester amides); poly(hydroxyalkanoates) (PHA), amino acids; PEG and/or alcohol groups, polycaprolactones, poly(L-lactide), poly(D,L-lactide), poly(D,L-lactide-co-PEG) block copolymers, poly(D,L-lactide-co-trimethylene carbonate), polyglycolides, poly(lactide-co-glycolide), polydioxanones, polyorthoesters, polyanhydrides, poly(glycolic acid-co-trimethylene carbonate), polyphosphoesters, polyphosphoester urethanes, poly(amino acids), polycyanoacrylates, poly(trimethylene carbonate), poly(imino carbonate), polycarbonates, polyurethanes, copoly(ether-esters) (e.g PEO/PLA), polyalkylene oxalates, polyphosphazenes, PHA-PEG, and any derivatives, analogs, homologues, salts, copolymers and combinations thereof.

In other embodiments, the polymers can be poly(glycerol sebacate); tyrosine-derived polycarbonates containing desaminotyrosyl-tyrosine alkyl esters such as, for example, desaminotyrosyl-tyrosine ethyl ester (poly(DTE carbonate)); and any derivatives, analogs, homologues, salts, copolymers and combinations thereof. In some embodiments, the polymers are selected such that they specifically exclude any one or any combination of any of the polymers taught herein.

In some embodiments, the polymers can be chemically connected to the agents by covalent bonds. In other embodiments, the polymers can be chemically connected to the agents by non-covalent bonds such as, for example, by ionic bonds, inter-molecular attractions, or a combination thereof. In other embodiments, the polymers can be physically connected to the agents. In other embodiments, the polymers can be chemically and physically connected with the agents. Examples of ionic bonding can include, but are not limited to, ionic bonding of an anionic site to a cationic site between polymers. In some embodiments, an anionic site can be bound to a quaternary amine. Examples of inter-molecular attractions include, but are not limited to, hydrogen bonding such as, for example, the permanent dipole interactions between hydroxyl, amino, carboxyl, amide, and sulfhydryl groups, and combinations thereof. Examples of physical connections can include, but are not limited to, interpenetrating networks and chain entanglement. The polymers can also be blended or mixed with the agents.

The Agents

Biobeneficial and Bioactive Agents

A “bioactive agent” is a moiety that can be combined with a polymer and provides a therapeutic effect, a prophylactic effect, both a therapeutic and a prophylactic effect, or other biologically active effect within a subject. Moreover, the bioactive agents of the present invention may remain linked to a portion of the polymer or be released from the polymer. A “biobeneficial agent” is an agent that can be combined with a polymer and provide a biological benefit within a subject without necessarily being released from the polymer.

In one example, a biological benefit may be that the polymer or coating becomes non-thrombogenic, such that protein absorption is inhibited or prevented to avoid formation of a thromboembolism; promotes healing, such that endothelialization within a blood vessel is not exuberant but rather forms a healthy and functional endothelial layer; or is non-inflammatory, such that the biobeneficial agent acts as a biomimic to passively avoid attracting monocytes and neutrophils, which could lead to an event or cascade of events that create inflammation.

A “diagnostic agent” is a type of bioactive agent that can be used, for example, in diagnosing the presence, nature, or extent of a disease or medical condition in a subject. In one embodiment, a diagnostic agent can be any agent that may be used in connection with methods for imaging an internal region of a patient and/or diagnosing the presence or absence of a disease in a patient. Diagnostic agents include, for example, contrast agents for use in connection with ultrasound imaging, magnetic resonance imaging (MRI), nuclear magnetic resonance (NMR), computed tomography (CT), electron spin resonance (ESR), nuclear medical imaging, optical imaging, elastography, and radiofrequency (RF) and microwave lasers. Diagnostic agents may also include any other agents useful in facilitating diagnosis of a disease or other condition in a patient, whether or not imaging methodology is employed.

Examples of biobeneficial agents include, but are not limited to, many of the polymers listed above such as, for example, carboxymethylcellulose; poly(alkylene glycols) such as, for example, PEG; poly(N-vinyl pyrrolidone); poly(acrylamide methyl propane sulfonic acid); poly(styrene sulfonate); sulfonated polysaccharides such as, for example, sulfonated dextran; sulfated polysaccharides such as, for example, sulfated dextran and dermatan sulfate; and glycosaminoglycans such as, for example, hyaluronic acid and heparin; and any derivatives, analogs, homologues, congeners, salts, copolymers and combinations thereof. In some embodiments, the biobeneficial agents can be prohealing such as, for example, poly(ester amides), elastin, silk-elastin, collagen, atrial natriuretic peptide (ANP); and peptide sequences such as, for example, those comprising Arg-Gly-Asp (RGD). In other embodiments, the biobeneficial agents can be non-thrombotics such as, for example, thrombomodulin; and antimicrobials such as, for example, the organosilanes. It is to be appreciated that one skilled in the art should recognize that some of the groups, subgroups, and individual biobeneficial agents may not be used in some embodiments of the present invention.

Examples of heparin derivatives include, but are not limited to, earth metal salts of heparin such as, for example, sodium heparin, potassium heparin, lithium heparin, calcium heparin, magnesium heparin, and low molecular weight heparin. Other examples of heparin derivatives include, but are not limited to, heparin sulfate, heparinoids, heparin-based compounds and heparin derivatized with hydrophobic materials.

Examples of hyaluronic acid derivates include, but are not limited to, sulfated hyaluronic acid such as, for example, O-sulphated or N-sulphated derivatives; esters of hyaluronic acid wherein the esters can be aliphatic, aromatic, arylaliphatic, cycloaliphatic, heterocyclic or a combination thereof; crosslinked esters of hyaluronic acid wherein the crosslinks can be formed with hydroxyl groups of a polysaccharide chain; crosslinked esters of hyaluronic acid wherein the crosslinks can be formed with polyalcohols that are aliphatic, aromatic, arylaliphatic, cycloaliphatic, heterocyclic, or a combination thereof; hemiesters of succinic acid or heavy metal salts thereof; quaternary ammonium salts of hyaluronic acid or derivatives such as, for example, the 0-sulphated or N-sulphated derivatives.

Examples of poly(alkylene glycols) include, but are not limited to, PEG, mPEG, poly(ethylene oxide), poly(propylene glycol)(PPG), poly(tetramethylene glycol), and any derivatives, analogs, homologues, congeners, salts, copolymers and combinations thereof. In some embodiments, the poly(alkylene glycol) is PEG. In other embodiments, the poly(alkylene glycol) is mPEG. In other embodiments, the poly(alkylene glycol) is poly(ethylene glycol-co-hydroxybutyrate).

The copolymers that may be used as biobeneficial agents include, but are not limited to, any derivatives, analogs, homologues, congeners, salts, copolymers and combinations of the foregoing examples of agents. Examples of copolymers that may be used as biobeneficial agents in the present invention include, but are not limited to, dermatan sulfate, which is a copolymer of D-glucuronic acid or L-iduronic acid and N-acetyl-D-galactosamine; poly(ethylene oxide-co-propylene oxide); copolymers of PEG and hyaluronic acid; copolymers of PEG and heparin; copolymers of PEG and hirudin; graft copolymers of poly(L-lysine) and PEG; copolymers of PEG and a poly(hydroxyalkanoate) such as, for example, poly(ethylene glycol-co-hydroxybutyrate); and, any derivatives, analogs, congeners, salts, or combinations thereof. In some embodiments, the copolymer that may be used as a biobeneficial agent can be a copolymer of PEG and hyaluronic acid, a copolymer of PEG and hirudin, and any derivative, analog, congener, salt, copolymer or combination thereof. In other embodiments, the copolymer that may be used as a biobeneficial agent is a copolymer of PEG and a poly(hydroxyalkanoate) such as, for example, poly(hydroxybutyrate); and any derivative, analog, congener, salt, copolymer or combination thereof.

The bioactive agents can be any moiety capable of contributing to a therapeutic effect, a prophylactic effect, both a therapeutic and prophylactic effect, or other biologically active effect in a mammal. The agent can also have diagnostic properties. The bioactive agents include, but are not limited to, small molecules, nucleotides, oligonucleotides, polynucleotides, amino acids, oligopeptides, polypeptides, and proteins. In one example, the bioactive agent inhibits the activity of vascular smooth muscle cells. In another example, the bioactive agent controls migration or proliferation of smooth muscle cells to inhibit restenosis.

Bioactive agents include, but are not limited to, antiproliferatives, antineoplastics, antimitotics, anti-inflammatories, antiplatelets, anticoagulants, antifibrins, antithrombins, antibiotics, antiallergics, antioxidants, and any prodrugs, metabolites, analogs, homologues, congeners, derivatives, salts and combinations thereof. It is to be appreciated that one skilled in the art should recognize that some of the groups, subgroups, and individual bioactive agents may not be used in some embodiments of the present invention.

Antiproliferatives include, for example, actinomycin D, actinomycin IV, actinomycin II, actinomycin X₁, actinomycin C₁, dactinomycin (COSMEGEN®, Merck & Co., Inc.), imatinib mesylate, and any prodrugs, metabolites, analogs, homologues, congeners, derivatives, salts and combinations thereof. Antineoplastics or antimitotics include, for example, paclitaxel (TAXOL®, Bristol-Myers Squibb Co.), docetaxel (TAXOTERE®, Aventis S. A.), methotrexate, azathioprine, vincristine, vinblastine, fluorouracil, doxorubicin hydrochloride (ADRIAMYCIN®, Pfizer, Inc.) and mitomycin (MUTAMYCIN®, Bristol-Myers Squibb Co.), midostaurin, and any prodrugs, metabolites, analogs, homologues, congeners, derivatives, salts and combinations thereof.

Antiplatelets, anticoagulants, antifibrin, and antithrombins include, for example, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors (ANGIOMAX®, Biogen, Inc.), and any prodrugs, metabolites, analogs, homologues, congeners, derivatives, salts and combinations thereof.

Cytostatic or antiproliferative agents include, for example, angiopeptin, angiotensin converting enzyme inhibitors such as captopril (CAPOTEN® and CAPOZIDE®, Bristol-Myers Squibb Co.), cilazapril or lisinopril (PRINIVIL® and PRINZIDE®, Merck & Co., Inc.); calcium channel blockers such as nifedipine; colchicines; fibroblast growth factor (FGF) antagonists, fish oil (omega 3-fatty acid); histamine antagonists; lovastatin (MEVACOR®, Merck & Co., Inc.); monoclonal antibodies including, but not limited to, antibodies specific for Platelet-Derived Growth Factor (PDGF) receptors; nitroprusside; phosphodiesterase inhibitors; prostaglandin inhibitors; suramin; serotonin blockers; steroids; thioprotease inhibitors; PDGF antagonists including, but not limited to, triazolopyrimidine; and nitric oxide, and any prodrugs, metabolites, analogs, homologues, congeners, derivatives, salts and combinations thereof. Antiallergic agents include, but are not limited to, pemirolast potassium (ALAMAST®, Santen, Inc.), and any prodrugs, metabolites, analogs, homologues, congeners, derivatives, salts and combinations thereof.

Other bioactive agents useful in the present invention include, but are not limited to, free radical scavengers; nitric oxide donors; rapamycin; methyl rapamycin; 42-Epi-(tetrazoylyl)rapamycin (ABT-578); 40-O-(2-hydroxy)ethyl-rapamycin (everolimus); tacrolimus; pimecrolimus; 40-O-(3-hydroxy)propyl-rapamycin; 40-O-[2-(2-hydroxy)ethoxy]ethyl-rapamycin; tetrazole containing rapamycin analogs such as those described in U.S. Pat. No. 6,329,386; estradiol; clobetasol; idoxifen; tazarotene; alpha-interferon; host cells such as epithelial cells; genetically engineered epithelial cells; dexamethasone; and, any prodrugs, metabolites, analogs, homologues, congeners, derivatives, salts and combinations thereof.

Free radical scavengers include, but are not limited to, 2,2′,6,6′-tetramethyl-1-piperinyloxy, free radical (TEMPO); 4-amino-2,2′,6,6′-tetramethyl-1-piperinyloxy, free radical (4-amino-TEMPO); 4-hydroxy-2,2′,6,6′-tetramethyl-piperidene-1-oxy, free radical (TEMPOL), 2,2′,3,4,5,5′-hexamethyl-3-imidazolinium-1-yloxy methyl sulfate, free radical; 16-doxyl-stearic acid, free radical; superoxide dismutase mimic (SODm) and any analogs, homologues, congeners, derivatives, salts and combinations thereof. Nitric oxide donors include, but are not limited to, S-nitrosothiols, nitrites, N-oxo-N-nitrosamines, substrates of nitric oxide synthase, diazenium diolates such as spermine diazenium diolate and any analogs, homologues, congeners, derivatives, salts and combinations thereof.

Examples of diagnostic agents include radioopaque materials and include, but are not limited to, materials comprising iodine or iodine-derivatives such as, for example, iohexal and iopamidol, which are detectable by x-rays. Other diagnostic agents such as, for example, radioisotopes, are detectable by tracing radioactive emissions. Other diagnostic agents may include those that are detectable by magnetic resonance imaging (MRI), ultrasound and other imaging procedures such as, for example, fluorescence and positron emission tomagraphy (PET). Examples of agents detectable by MRI are paramagnetic agents, which include, but are not limited to, gadolinium chelated compounds. Examples of agents detectable by ultrasound include, but are not limited to, perflexane. Examples of fluorescence agents include, but are not limited to, indocyanine green. Examples of agents used in diagnostic PET include, but are not limited to, fluorodeoxyglucose, sodium fluoride, methionine, choline, deoxyglucose, butanol, raclopride, spiperone, bromospiperone, carfentanil, and flumazenil.

In some embodiments, a combination of agents can be applied, as taught herein, within controlled volumes within a medical device, on a medical device, or positioned within a controlled volume at a predetermined region on the device or within a coating on the device. In some embodiments, the agent combination includes everolimus and clobetasol. In other embodiments, the agent combination includes tacrolimus and rapamycin. In other embodiments, the agent combination includes tacrolimus and everolimus. In other embodiments, the agent combination can include rapamycin and paclitaxel. In other embodiments, the agent combination can include an anti-inflammatory such as, for example, a corticosteroid and an antiproliferative such as, for example, everolimus. In some embodiments, the agent combinations can provide synergistic effects for preventing or inhibiting conditions such as, for example, restenosis that may occur through use of a stent.

Plasticizing Agents

The terms “plasticizer” and “plasticizing agent” can be used interchangeably in the present invention, and refer to any agent, including any agent described above, where the agent can be added to a polymeric composition to modify the mechanical properties of the composition or a product formed from the composition. Plasticizers can be added, for example, to reduce crystallinity, lower the glass-transition temperature (T_{g}), or reduce the intermolecular forces between polymers, with design goals that may include, but are not limited to, enhancing mobility between polymer chains in the composition. The mechanical properties that are modified include, but are not limited to, Young's modulus, impact resistance (toughness), tensile strength, and tear strength. Impact resistance, or “toughness,” is a measure of energy absorbed during fracture of a polymer sample of standard dimensions and geometry when subjected to very rapid impact loading. Toughness can be measured using Charpy and Izod impact tests to assess the brittleness of a material.

A plasticizer can be monomeric, polymeric, co-polymeric, or a combination thereof, and can be combined with a polymeric composition in the same manner as described above for the biobeneficial and bioactive agents. Plasticization and solubility are analogous in the sense that selecting a plasticizer involves considerations similar to selecting a solvent such as, for example, polarity. Furthermore, plasticization can also be provided through covalent bonding by changing the molecular structure of the polymer through copolymerization.

Examples of plasticizing agents include, but are not limited to, low molecular weight polymers such as single-block polymers, multi-block polymers, and copolymers; oligomers such as ethyl-terminated oligomers of lactic acid; small organic molecules; hydrogen bond forming organic compounds with and without hydroxyl groups; polyols such as low molecular weight polyols having aliphatic hydroxyls; alkanols such as butanols, pentanols and hexanols; sugar alcohols and anhydrides of sugar alcohols; polyethers such as poly(alkylene glycols); esters such as citrates, phthalates, sebacates and adipates; polyesters; aliphatic acids; proteins such as animal proteins and vegetable proteins; oils such as, for example, the vegetable oils and animal oils; silicones; acetylated monoglycerides; amides; acetamides; sulfoxides; sulfones; pyrrolidones; oxa acids; diglycolic acids; and any analogs, derivatives, copolymers and combinations thereof.

In some embodiments, the plasticizers include, but are not limited to other polyols such as, for example, caprolactone diol, caprolactone triol, sorbitol, erythritol, glucidol, mannitol, sorbitol, sucrose, and trimethylol propane. In other embodiments, the plasticizers include, but are not limited to, glycols such as, for example, ethylene glycol, diethylene glycol, triethylene glycol, tetraethylene glycol, propylene glycol, butylene glycol, 1,2-butylene glycol, 2,3-butylene glycol, styrene glycol, pentamethylene glycol, hexamethylene glycol; glycol-ethers such as, for example, monopropylene glycol monoisopropyl ether, propylene glycol monoethyl ether, ethylene glycol monoethyl ether, and diethylene glycol monoethyl ether; and any analogs, derivatives, copolymers and combinations thereof.

In other embodiments, the plasticizers include, but are not limited to esters such as glycol esters such as, for example, diethylene glycol dibenzoate, dipropylene glycol dibenzoate, triethylene glycol caprate-caprylate; monostearates such as, for example, glycerol monostearate; citrate esters; organic acid esters; aromatic carboxylic esters; aliphatic dicarboxylic esters; fatty acid esters such as, for example, stearic, oleic, myristic, palmitic, and sebacic acid esters; triacetin; poly(esters) such as, for example, phthalate polyesters, adipate polyesters, glutate polyesters, phthalates such as, for example, dialkyl phthalates, dimethyl phthalate, diethyl phthalate, isopropyl phthalate, dibutyl phthalate, dihexyl phthalate, dioctyl phthalate, diisononyl phthalate, and diisodecyl phthalate; sebacates such as, for example, alkyl sebacates, dimethyl sebacate, dibutyl sebacate; hydroxyl-esters such as, for example, lactate, alkyl lactates, ethyl lactate, butyl lactate, allyl glycolate, ethyl glycolate, and glycerol monostearate; citrates such as, for example, alkyl acetyl citrates, triethyl acetyl citrate, tributyl acetyl citrate, trihexyl acetyl citrate, alkyl citrates, triethyl citrate, and tributyl citrate; esters of castor oil such as, for example, methyl ricinolate; aromatic carboxylic esters such as, for example, trimellitic esters, benzoic esters, and terephthalic esters; aliphatic dicarboxylic esters such as, for example, dialkyl adipates, alkyl allylether diester adipates, dibutoxyethoxyethyl adipate, diisobutyl adipate, sebacic esters, azelaic esters, citric esters, and tartaric esters; and fatty acid esters such as, for example, glycerol, mono- di- or triacetate, and sodium diethyl sulfosuccinate; and any analogs, derivatives, copolymers and combinations thereof.

In other embodiments, the plasticizers include, but are not limited to ethers and polyethers such as, for example, poly(alkylene glycols) such as poly(ethylene glycols) (PEG), poly(propylene glycols), and poly(ethylene/propylene glycols); low molecular weight poly(ethylene glycols) such as, for example, PEG 400 and PEG 6000; PEG derivatives such as, for example, methoxy poly(ethylene glycol) (mPEG); and ester-ethers such as, for example, diethylene glycol dibenzoate, dipropylene glycol dibenzoate, and triethylene glycol caprate-caprylate; and any analogs, derivatives, copolymers and combinations thereof.

In other embodiments, the plasticizers include, but are not limited to, amides such as, for example, oleic amide, erucic amide, and palmitic amide; alkyl acetamides such as, for example, dimethyl acetamide and dimethyl formamide; sulfoxides such as for example, dimethyl sulfoxide; pyrrolidones such as, for example, n-methyl pyrrolidone; sulfones such as, for example, tetramethylene sulfone; acids such as, for example, oxa monoacids, oxa diacids such as 3,6,9-trioxaundecanedioic acid, polyoxa diacids, ethyl ester of acetylated citric acid, butyl ester of acetylated citric acid, capryl ester of acetylated citric acid, and diglycolic acids such as dimethylol propionic acid; and any analogs, derivatives, copolymers and combinations thereof.

In other embodiments, the plasticizers can be vegetable oils including, but not limited to, epoxidized soybean oil; linseed oil; castor oil; coconut oil; fractionated coconut oil; epoxidized tallates; and esters of fatty acids such as stearic, oleic, myristic, palmitic, and sebacic acid. In other embodiments, the plasticizers can be essential oils including, but not limited to, angelica oil, anise oil, arnica oil, aurantii aetheroleum, valerian oil, basilici aetheroleum, bergamot oil, savory oil, bucco aetheroleum, camphor, cardamomi aetheroleum, cassia oil, chenopodium oil, chrysanthemum oil, cinae aetheroleum, citronella oil, lemon oil, citrus oil, costus oil, curcuma oil, carlina oil, elemi oil, tarragon oil, eucalyptus oil, fennel oil, pine needle oil, pine oil, filicis, aetheroleum, galbanum oil, gaultheriae aetheroleum, geranium oil, guaiac wood oil, hazelwort oil, iris oil, hypericum oil, calamus oil, camomile oil, fir needle oil, garlic oil, coriander oil, carraway oil, lauri aetheroleum, lavender oil, lemon grass oil, lovage oil, bay oil, lupuli strobuli aetheroleum, mace oil, marjoram oil, mandarine oil, melissa oil, menthol, millefolii aetheroleum, mint oil, clary oil, nutmeg oil, spikenard oil, clove oil, neroli oil, niaouli, olibanum oil, ononidis aetheroleum, opopranax oil, orange oil, oregano oil, orthosiphon oil, patchouli oil, parsley oil, petit-grain oil, peppermint oil, tansy oil, rosewood oil, rose oil, rosemary oil, rue oil, sabinae aetheroleum, saffron oil, sage oil, sandalwood oil, sassafras oil, celery oil, mustard oil, serphylli aetheroleum, immortelle oil, fir oil, teatree oil, terpentine oil, thyme oil, juniper oil, frankincense oil, hyssop oil, cedar wood oil, cinnamon oil, and cypress oil; and other oils such as, for example, fish oil; and, any analogs, derivatives, copolymers and combinations thereof.

The molecular weights of the plasticizers can vary. In some embodiments, the molecular weights of the plasticizers range from about 10 Daltons to about 50,000 Daltons; from about 25 Daltons to about 25,000 Daltons; from about 50 Daltons to about 10,000 Daltons; from about 100 Daltons to about 5,000 Daltons; from about 200 Daltons to about 2500 Daltons; from about 400 Daltons to about 1250 Daltons; and any range therein. In other embodiments, the molecular weights of the plasticizers range from about 400 Daltons to about 4000 Daltons; from about 300 Daltons to about 3000 Daltons; from about 200 Daltons to about 2000 Daltons; from about 100 Daltons to about 1000 Daltons; from about 50 Daltons to about 5000 Daltons; and any range therein. The molecular weights are taught herein as a number average molecular weight.

The amount of plasticizer used in the present invention, can range from about 0.001% to about 70%; from about 0.01% to about 60%; from about 0.1% to about 50%; from about 0.1% to about 40%; from about 0.1% to about 30%; from about 0.1% to about 25%; from about 0.1% to about 20%; from about 0.1% to about 10%; from about 0.4% to about 40%; from about 0.6% to about 30%; from about 0.75% to about 25%; from about 1.0% to about 20%; and any range therein, as a weight percentage based on the total weight of the polymer and agent or combination of agents.

It should be appreciated that any one or any combination of the plasticizers described above can be used in the present invention. For example, the plasticizers can be combined to obtain the desired finction. In some embodiments, a secondary plasticizer is combined with a primary plasticizer in an amount that ranges from about 0.001% to about 20%; from about 0.01% to about 15%; from about 0.05% to about 10%; from about 0.75% to about 7.5%; from about 1.0% to about 5%, or any range therein, as a weight percentage based on the total weight of the polymer any agent or combination of agents.

It should also be appreciated that the plasticizers can be combined with other active agents to obtain other desired functions such as, for example, an added therapeutic, prophylactic, and/or diagnostic function. In some embodiments, the plasticizers can be linked to other agents through ether, amide, ester, orthoester, anhydride, ketal, acetal, carbonate, and all-aromatic carbonate linkages, which are discussed in more detail below.

In some embodiments, the agents can be chemically connected to a polymer by covalent bonds. In other embodiments, the agents can be chemically connected to a polymer by non-covalent bonds such as, for example, by ionic bonds, inter-molecular attractions, or a combination thereof. In other embodiments, the agents can be physically connected to a polymer. In other embodiments, the agents can be chemically and physically connected with a polymer.

Examples of ionic bonding can include, but are not limited to, ionic bonding of an anionic agent to a cationic site on a polymer or a cationic agent to an anionic site on a polymer. In some embodiments, an anionic agent can be bound to a quaternary amine on a polymer. In other embodiments, an agent with a quaternary amine can be bound to an anionic site on a polymer. Examples of inter-molecular attractions include, but are not limited to, hydrogen bonding such as, for example, the permanent dipole interactions between hydroxyl, amino, carboxyl, and sulfhydryl groups, and combinations thereof. Examples of physical connections can include, but are not limited to, interpenetrating networks and chain entanglement. The agents can also be blended or mixed with the compositions.

In some embodiments, the agents have a reactive group that can be used to link the agents to the polymer. Examples of reactive groups include, but are not limited to, hydroxyl, acyl, amino, amido, and sulfhydryl groups. In some embodiments, the agents can be released or can separate from the polymer composition. In other embodiments, the agents can be biobeneficial, bioactive, diagnostic, plasticizing, or have a combination of these characteristics.

In some embodiments, the molecular weight of an agent should be at or below about 40,000 Daltons, or any range therein, to ensure elimination of the agent from a mammal. In one embodiment, the molecular weight of the agent ranges from about 300 Daltons to about 40,000 Daltons, from about 8,000 Daltons to about 30,000 Daltons, from about 10,000 Daltons to about 20,000 Daltons, or any range therein. If upon release, the biobeneficial agent is rapidly broken down in the body, then the molecular weight of the agent could be greater than about 40,000 Daltons without compromising patient safety. The molecular weights as taught herein are a number average molecular weight.

It should also be appreciated that the agents of the present invention can have properties that are biobeneficial, bioactive, diagnostic, plasticizing or a combination thereof. For example, classification of an agent as a biobeneficial agent does not preclude the use of that agent as a bioactive agent, diagnostic agent and/or plasticizing agent. Likewise, classification of an agent as a bioactive agent does not preclude the use of that agent as a diagnostic agent, biobeneficial agent and/or plasticizing agent. Furthermore, classification of an agent as a plasticizing agent does not preclude the use of that agent as a biobeneficial agent, bioactive agent, and/or diagnostic agent. It should also be appreciated that any of the foregoing agents can be combined with the compositions such as, for example, in the form of a medical device or a coating for a medical device. By way of a non-limiting example, a stent coated with the compositions of the invention can contain paclitaxel, docetaxel, rapamycin, methyl rapamycin, ABT-578, everolimus, or clobetasol.

Concentrations of Agents

The agents of the present invention can be added in combination to obtain other desired functions of the polymeric compositions. The amounts of the agents that compose the polymeric compositions vary according to a variety of factors including, but not limited to, the biological activity of the agent; the age, body weight, response, or the past medical history of the subject; the type of atherosclerotic disease; the presence of systemic diseases such as, for example, diabetes; the pharmacokinetic and pharmacodynamic effects of the agents or combination of agents; and the design of the compositions for sustained release of the agents. Factors such as these are routinely considered by one of skill in the art when administering an agent to a subject.

It is to be appreciated that the design of a composition for the sustained release of agents can be dependent on a variety of factors such as, for example, the therapeutic, prophylactic, ameliorative or diagnostic needs of a patient. In some embodiments, the agent can comprise an antiproliferative and should have a sustained release ranging from about 1 week to about 10 weeks, from about 2 weeks to about 8 weeks, from about 3 weeks to about 7 weeks, from about 4 weeks to about 6 weeks, and any range therein. In other embodiments, the agent can comprise an anti-inflammatory and should have a sustained release ranging from about 6 hours to about 3 weeks, from about 12 hours to about 2 weeks, from about 18 hours to about 10 days, from about 1 day to about 7 days, from about 2 days to about 6 days, or any range therein. In general, the sustained release should range from about 4 hours to about 12 weeks; alternatively, from about 6 hours to about 10 weeks; or from about 1 day to about 8 weeks.

Effective amounts, for example, may be extrapolated from in vitro or animal model systems. In some embodiments, the agent or combination of agents have a concentration that ranges from about 0.001% to about 75%; from about 0.01% to about 70%; from about 0.1% to about 60%; from about 0.25% to about 60%; from about 0.5% to about 50%; from about 0.75% to about 40%; from about 1.0% to about 30%; from about 2% to about 20%; and, any range therein, where the percentage is based on the total weight of the polymer and agent or combination of agents.

Forming a Medical Article

The agent can be monodispersed and localized in an implant during a process of forming the implant, and the localization can be beneficial for a variety of reasons such as, for example, use of less agent in select regions; use of a preferred agent in select regions such as, for example, an agent with desired potency or faster leaching rate; modification of mechanical properties of select regions of an implant; leaching of less agent for elimination by a subject; and combinations thereof. In some embodiments, there may be no agent in the regions outside of the high-strain regions in an implant. In other embodiments, there may be less agent in the regions outside of the high-strain regions in an implant. In embodiments where less agent is desired in the regions outside of the high-strain regions, the amount of agent in the regions outside of the high-strain regions can have 2%, 5%, 10%, 15%, 20%, 30%, 40%, 50%, 60%, 70%, 80%, 90%, 95%, or any range therein, less agent than the high-strain regions.

Processes for forming a medical article include, but are not limited to, casting, molding, coating, and combinations thereof. In some embodiments, the implant is formed in a casting process, and the mechanical properties of the high-strain regions of the implant are controlled by concentrating the agent in the high-strain regions, by using different agents in the high-strain regions, by using agents only in the high-strain regions, or a combination thereof. Casting an implant involves pouring a liquid polymeric composition into a mold. In one embodiment, the localization of an agent in an implant during such casting can be obtained by varying the amount and/or type of agent in the polymeric composition during pouring as desired such that the agent becomes localized in the formed implant.

In other embodiments, the implant is formed in a molding process, which includes, but is not limited to, compression molding, extrusion molding, injection molding, and foam molding. The mechanical properties of the high-strain regions of the implant are controlled by concentrating the agent in the high-strain regions, by using different agents in the high-strain regions, by using agents only in the high-strain regions, or a combination thereof.

In compression molding, solid polymeric materials are added to a mold and pressure and heat are applied until the polymeric material conforms to the mold. The solid form may require additional processing to obtain the final product in a desired form. The solid polymeric materials can be in the form of particles that can vary in mean diameter from about 1 nm to about 1 cm, from about 1 nm to about 10 mm, from about 1 nm to about 1 mm, from about 1 nm to about 100 nm, or any range therein. In one embodiment, the localization of agents in an implant during such compression molding can be obtained by varying the amount and/or type of agent in the solid polymeric materials while adding the solid polymeric materials to the mold as desired such that the agent becomes localized in the formed implant.

In extrusion molding, solid polymeric materials are added to a continuous melt that is forced through a die and cooled to a solid form. The solid form may require additional processing to obtain the final product in a desired form. The solid polymeric materials can be in the form of particles that can vary in mean diameter from about 1 nm to about 1 cm, from about 1 nm to about 10 mm, from about 1 nm to about 1 mm, from about 1 nm to about 100 nm, or any range therein. In one embodiment, the localization of agent in an implant during such extrusion molding can be obtained by varying the amount and/or type of agent in the solid polymeric materials while adding the solid polymeric materials to the extrusion mold as desired such that the agent becomes localized in the formed implant.

In injection molding, solid polymeric materials are added to a heated cylinder, softened and forced into a mold under pressure to create a solid form. The solid form may require additional processing to obtain the final product in a desired form. The solid polymeric materials can be in the form of particles that can vary in mean diameter from about 1 nm to about 1 cm, from about 1 nm to about 10 mm, from about 1 nm to about 1 mm, from about 1 nm to about 100 nm, or any range therein. In one embodiment, the localization of agent in an implant during such injection molding can be obtained by varying the amount and/or type of agent in the solid polymeric materials while adding the solid polymeric materials to the injection mold as desired such that the agent becomes localized in the formed implant.

In foam molding, blowing agents are used to expand and mold solid polymeric materials into a desired form, and the solid polymeric materials can be expanded to a volume ranging from about two to about 50 times their original volume. The polymeric material can be pre-expanded using steam and air and then formed in a mold with additional steam; or mixed with a gas to form a polymer/gas mixture that is forced into a mold of lower pressure. The solid form may require additional processing to obtain the final product in a desired form. The solid polymeric materials can be in the form of particles that can vary in mean diameter from about 1 nm to about 1 cm, from about 1 nm to about 10 mm, from about 1 nm to about 1 mm, from about 1 nm to about 100 nm, or any range therein. In one embodiment, the localization of agent in an implant during such foam molding can be obtained by varying the amount and/or type of agent in the solid polymeric materials while adding the solid polymeric materials to the foam mold as desired such that the agent becomes localized in the formed implant.

In other embodiments, a stent is formed by injection molding or extrusion of a tube followed by cutting a pattern of a stent into the tube. In these embodiments, a mixture of polymer and agent can be added prior to injection molding or extrusion or, in the alternative, the agent can be absorbed by the stent after the stent has been formed.

Forming a Coating

In some embodiments of the invention, the compositions are in the form of coatings for medical devices such as, for example, a balloon-expandable stent or a self-expanding stent. There are many coating configurations within the scope of the present invention, and each configuration can include any number and combination of layers. In some embodiments, the coatings of the present invention can comprise one or a combination of the following four types of layers:

(a) an agent layer, which may comprise a polymer and an agent or, alternatively, a polymer free agent;

(b) an optional primer layer, which may improve adhesion of subsequent layers on the implantable substrate or on a previously formed layer;

(c) an optional topcoat layer, which may serve as a way of controlling the rate of release of an agent; and

(d) an optional biocompatible finishing layer, which may improve the biocompatibility of the coating.

In one embodiment, the agent layer can be applied directly to at least a part of an implantable substrate as a pure agent to serve as a reservoir for at least one bioactive agent. In another embodiment, the agent can be combined with a biodegradable polymer as a matrix, wherein agent may or may not be bonded to the polymer. In another embodiment, the optional primer layer can be applied between the implantable substrate and the agent layer to improve adhesion of the agent layer to the implantable substrate and can optionally comprise an agent. In another embodiment, a pure agent layer can be sandwiched between layers comprising biodegradable polymer. In another embodiment, the optional topcoat layer can be applied over at least a portion of the agent layer to serve as a membrane to control the rate of release of the bioactive agent and can optionally comprise agent. In another embodiment, the biocompatible finishing layer can also be applied to increase the biocompatibility of the coating by, for example, increasing acute hemocompatibility and can also comprise an agent.

The inventive compositions can be used for one or any combination of layers, and a layer may comprise one or more controlled volumes such as, for example, selectively-placed droplets that serve as agents positioned within a controlled volume at a predetermined region on the device or within the coating. In some embodiments, any of the polymers taught herein can be used as one of the layers or can be blended or crosslinked with the compositions in the embodiments taught herein.

In some embodiments, the methods of the present invention can be used to coat a medical device with layers formed from controlled volumes such as, for example, droplets, using the methods of the present invention. In one example, the droplets can be formed only from a pure agent or an agent dispersed within a solvent. In another example, the droplets can be formed from a combination of an agent and a polymer. In another example, the droplets can be formed from agents encapsulated by a polymer and, in this example, the encapsulation can provide controlled release of the agent, protect the agent to improve shelf-life, or a combination thereof. In another example, the droplets can be formed from any one or any combination of components and then coated with a topcoat layer to provide controlled release of the agent, protect the agent to improve shelf-life, or a combination thereof.

In another example, the droplets can be formed and applied as a suspension with a coating composition, and the coating composition can be applied using traditional coating methods such as, for example, spraying and dipping. In another example, the droplets can be formed and disperse in a polymeric composition used to form the structure of a medical device. In another example, the droplets can be formed in various sizes, wherein the sizes can vary due to the amount of agent, amount of encapsulating polymer, or a combination thereof. In another example, the droplets can be sandwiched between one or more other layers that can be formed from droplets or more traditional coating techniques such as, for example, spraying or dipping.

In many embodiments, each layer can be applied to an implantable substrate by any method including, but not limited to, dipping, spraying, pouring, brushing, spin-coating, roller coating, meniscus coating, powder coating, inkjet-type application, controlled-volume application such as drop-on-demand, or a combination thereof. In one example, at least one of the layers can be formed on a stent by dissolving one or more biodegradable polymers, optionally with a non-biodegradable polymer, in one or more solvents, and either (i) spraying the solution on the stent or (ii) dipping the stent in the solution. In this example, a dry coating of biodegradable polymer may be formed on the stent when the solvent evaporates.

In other embodiments, a medical device, such as a stent, can be coated with a polymeric material using methods that may include sputtering and gas-phase polymerization. Sputtering is a method that includes placing a polymeric material target in an environment that is conducive applying energy to the polymeric material and sputtering the polymeric material from the target to the device to form a coating of the polymeric material on the device. Similarly, a gas-phase polymerization method includes applying energy to a monomer in the gas phase within an environment that is conducive to formation of a polymer from the monomer in the gas phase, and wherein the polymer formed coats the device.

Sputtering and gas-phase polymerization have shortcomings similar to those that can be present in dip-coating and spray-coating techniques. The shortcomings include the lack of control of the geometrical patterns in which the medical device can be coated, in addition to the limited selection of polymers that can be used to coat the device. Furthermore, coating a device with a polymer and drug combination can affect the outcome of the coating process, such as, for example, by creating drug degradation during the coating application.

FIGS. 3a and 3b illustrate a sandwiched-coating design according to some embodiments of the present invention. FIG. 3a illustrates a cross-section of a stent strut 301 in which the abluminal surface 302 includes a first layer 303 containing agent B applied to the abluminal surface 302 and a second layer 304 containing agent A applied on the layer 303 containing agent B. Each of the layers can be formed by any one or any combination of the methods described above and can be applied to the entire stent or select regions of the stent. In one example, the first layer 303 can be formed entirely of controlled-volume droplets, and the second layer 304 can be a blend of agent B with a select polymer. In another example, the first layer 303 can be a blend of agent B with a select polymer, and the second layer 304 can be formed entirely of controlled-volume droplets. In another example, the first layer 303 and the second layer 304 can be formed entirely of controlled-volume droplets. FIG. 3b illustrates a cross-section of the stent strut 301 in which the first layer 303 and the second layer 304 are coated by a third layer 305. The third layer 305 can contain any composition taught herein such as, for example, a rate-controlling biodegradable polymer to assist in controlling the rate of release of the agents or a biobeneficial layer.

FIG. 4 illustrates a checkerboard-type coating design by showing a top view of the abluminal surface of a stent that was coated in sections according to some embodiments of the present invention. The process of coating the abluminal surface 401 of the stent in sections 402 can occur simultaneously or as a series of coating steps. Each section of the checkerboard-type coating design can have sections 402 that are individually applied as controlled-volume droplets or applied as a plurality of controlled-volume droplets within each section 402. In one example, each of the sections 402 contain a single agent. In another example, each section 402 contain more than one agent. In another example, each section 402 contains agent concentrations that are similar or equal. In another example, each section 402 contains agent concentrations that vary depending on the agent.

In another example, each section 402 contains agent concentrations that vary depending on the desired release profile of the agent, which may be controlled, for example, through the addition of a biodegradable polymer that is combined with the agent. In another example, each section 402 contains agent concentrations that vary depending on the area of the stent in which the agent is located. In another example, each section 402 has a similar or equal thickness. In another example, each section 402 can vary in thickness due to any one or any combination of the above factors.

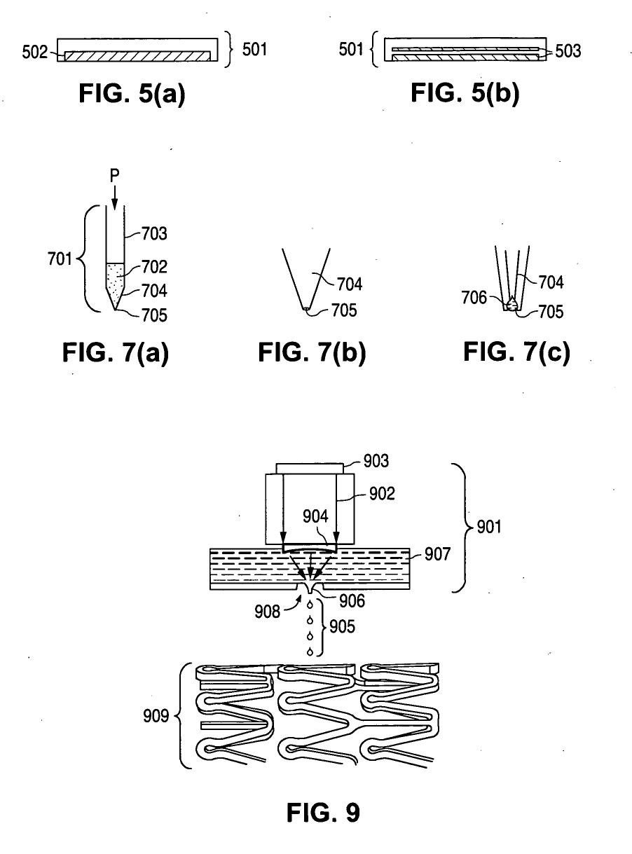

FIGS. 5a and 5b illustrate an engraved-type coating design by showing a top view of the abluminal surface of a stent with engravings according to some embodiments of the present invention. The engravings can be in any shape, size or form such as, for example, channels or pits. FIG. 5a shows a single channel 502 on the abluminal surface 501 of the stent, and FIG. 5b shows a parallel track-type coating design 503 on the abluminal surface 501 of the stent.

In one example, a channel width can range from about 0.0005 inches to about 0.005 inches. In another example, the channel width can range from about 0.001 inches to about 0.004 inches. In another example, the channel width can range from about 0.001 inches to about 0.002 inches. In another example, there can be a single pit. In another example, the engravings can be continuous on the abluminal surface on each strut of the stent such as, for example, a continuous channel. In another example, the engravings can be discontinuous and placed in select regions on the abluminal surface of the stent. In another example, the stent can have a combination of any shape engravings such as, for example, a combination of channels and pits. The pits and channels can be formed using any method known to one of skill in the art such as, for example, laser cutting, extruding, or molding.

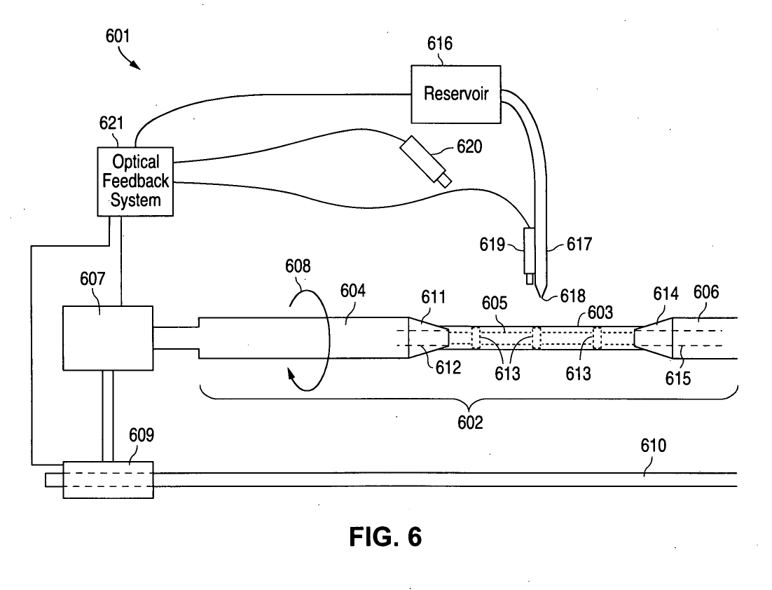

FIG. 6 illustrates a stent coating apparatus according to some embodiments of the present invention. The apparatus 601, including a stent mandrel fixture 602 for supporting the stent 603, is illustrated to include a support member 604, a mandrel 605, and an optional lock member 606 (e.g., if the stent 603 can be supported by the mandrel 605 itself). The support member 604 can connect to a motor 607 so as to provide rotational motion about the longitudinal axis of the stent 603, as depicted by arrow 608, during a coating process. Another motor 609 can also be provided for moving the support member 604 in a linear direction, back and forth, along a rail 610.

The support member 604 includes a coning end portion 611, tapering inwardly. In accordance with one embodiment of the invention, the mandrel 605 can be permanently affixed to coning end portion 611. Alternatively, the support member 604 can include a bore 612 for receiving a first end of the mandrel 605. The first end of mandrel 605 can be threaded to screw into the bore 612 or, alternatively, can be retained within the bore 612 by a friction fit. The bore 612 should be deep enough so as to allow the mandrel 605 to securely mate with the support member 604. The depth of the bore 612 can also be over-extended so as to allow a significant length of the mandrel 605 to penetrate or screw into the bore 612. The bore 612 can also extend completely through the support member 604. This would allow the length of the mandrel 605 to be adjusted to accommodate stents of various sizes. The mandrel 605 may also include a plurality of ridges 613 that add rigidity and support to the stent 603 during the coating process. The ridges 613 have a diameter of slightly less than the inner diameter of stent 603. While three ridges 613 are shown, it will be appreciated by one of ordinary skill in the art that additional, fewer, or no ridges may be present and any ridges may be evenly or unevenly spaced. In some embodiments, a stiff mandrel 605 can help to improve the precision of the coating process, since a minimum amount of run-out in imaging and application of coating compositions is usually preferred.

The lock member 606 includes a coning end portion 614 tapering inwardly. A second end of the mandrel 605 can be permanently affixed to the lock member 606 if the first end is disengagable from the support member 604. Alternatively, in accordance with another embodiment, the mandrel 605 can have a threaded second end for screwing into a bore 615 of the lock member 606. The bore 615 can be of any suitable depth that would allow the lock member 606 to be incrementally moved closer to the support member 604. The bore 615 can also extend completely through the lock member 606. Accordingly, stents 603 of any length can be securely pinched between the support and the lock members 604 and 606. In accordance with yet another embodiment, a non-threaded second end and the bore 615 combination is employed such that the second end can be press-fitted or friction-fitted within the bore 615 to prevent movement of the stent 603 on the stent mandrel fixture 602.

Positioned a distance from the stent 603 (e.g., above the stent 603) is a reservoir 616 holding a coating composition to be applied to the stent 603. The reservoir 616 is in fluid communication with an ejector 617 having an aperture 618. The ejector 617 is also positioned a distance from the stent 603 (e.g., above, below and/or at an angle to the stent 603). A source of pressure can be used to dispense the coating compositions such as, for example, hydrostatic pressure, hydraulic pressure, pneumatic pressure, capillary pressure, or any other source of pressure known to one of skill in the art. For example, in some embodiments, a transducer can be disposed within the ejector 617 to converts electrical energy into vibrational energy in the form of sound or ultrasound. The sound or ultrasound is referred herein to as “acoustic” energy and ejects controlled-volumes such as, for example, drops of the coating composition, from the aperture 618 onto the stent 603. In an embodiment of the invention, each acoustic pulse from the transducer can dispense a single drop from the aperture 618.

The reservoir 616 dispenses the coating composition to the ejector 617, which ejects it through the aperture 618. The aperture 618 has a small opening ranging from about 50 μm to about 250 μm in diameter and, therefore, the coating composition will not exit the aperture 618 due to surface tension of the coating composition unless the transducer is activated. In some embodiments, a coating can be used to control the surface energy of the aperture 618 such, for example, TEFLON can be used to provide a low surface energy coating. The transducer can be adjusted to control the rate of coating dispensed so that certain sections of the stent 603 can receive more coating than others.

The ejector 617 can be aligned with each individual stent strut. The coating flows into the ejector 617 and is ejected from the aperture 618 by the transducer onto the stent strut controllably to limit the coating to just the abluminal surface stent strut, which is an advantage over spraying and immersion techniques. In some embodiments, the sidewalls of the stent struts can be partially coated. In other embodiments, partial coating of sidewalls can be incidental or intentional.

Coupled to the ejector 617 can be a first imaging device 619 that images the stent 603 before and/or after the coating composition has been applied to a portion of the stent 603. The first imaging device 619, along with a second imaging device 620 located a distance from the stent 603, are both communicatively coupled to an optical feedback system 621 using wired or wireless techniques. The reservoir 616 may also be communicatively coupled to the optical feedback system 621 using wired or wireless techniques. Based on the imagery provided by the imaging devices 619 and 620, the optical feedback system 621 controls movement of stent 603 using the motors 607 and 609 to keep the aperture 618 aligned with the stent struts as required.

During operation of the stent coating apparatus 601, the optical feedback system 621 causes the imaging device 620 to image the full surface of the stent 603 as the feedback system 621 causes the motor 607 to rotate the stent 603. After the initial imaging, the optical feedback system 621, using the imaging device 619, aligns the aperture 618 with a stent strut by causing the motors 607 and 609 to rotate and translate the stent 603 until alignment is achieved. The optical feedback system 621 then causes the transducer to dispense the coating substance through the aperture 618 by emitting acoustic energy towards coating composition located in the aperture 618.

As the coating substance is dispensed, the optical feedback system 621 causes the motors 607 and 609 to rotate and translate the stent 603 in relation to the aperture 618 so as to position remaining desired sections of the stent strut along the aperture 618, thereby causing the desired abluminal surfaces of the strut to be coated. In one example, the entire abluminal surface of the stent is coated with the composition. In another example, select areas are coated with a first composition, and the process is repeated with one or more additional compositions. In another example, the process is performed using plurality of apertures 618 with a corresponding plurality of compositions to coat desired surfaces of the stent simultaneously.

After a portion of the stent strut has been coated, the optical feedback system 621 causes the transducer to cease dispensing the coating composition and causes the imaging device 619 to image the stent strut to determine if the strut has been adequately coated. This determination can be made by measuring the difference in color and/or reflectivity of the stent strut before and after the coating process. If the strut has been adequately coated, then the optical feedback system 621 causes the motors 607 and 609 to rotate and translate the stent 603 so that the aperture 618 is aligned with an uncoated stent 603 section and the above process is then repeated. If the stent strut is not coated adequately, then the optical feedback system 621 causes the motors 607 and 609 to rotate and translate the stent 603 and the transducer to dispense the coating composition to recoat the stent strut. In another embodiment of the invention, the optical feedback system 621 can cause checking and recoating of the stent 603 after the entire stent 603 goes through each coating pass.

In an embodiment of the invention, the imaging devices 619 and 620 can include charge coupled devices (CCDs) or complementary metal oxide semiconductor (CMOS) devices. In an embodiment of the invention, the imaging devices 619 and 620 can be combined into a single imaging device. Further, it will be appreciated by one of ordinary skill in the art that placement of the imaging devices 619 and 620 can vary as long as they have an acceptable view of the stent 603. In addition, one of ordinary skill in the art will realize that the stent mandrel fixture 602 can take any form or shape as long as it is capable of securely holding the stent 603 in place.

Accordingly, the embodiments of the invention enable the fine coating of specific surfaces of the stent 603, thereby avoiding coating defects that can occur with spray coating and immersion coating methods and limiting the coating to only the abluminal surface and/or sidewalls of the stent 603. Application of the coating in gaps between the stent struts can be partially or completely avoided using the techniques taught herein.

In many embodiments, the coating can be include depots or patterns as described in U.S. Pat. No. 6,395,326, which is incorporated herein by reference. In some embodiments, preselected geometrical patterns can be deposited by moving a dispenser assembly, such as the acoustic ejector assembly, along a predetermined path while depositing the composition onto a stationary medical device such as, for example, a prosthesis or a stent. In other embodiments, the preselected geometrical pattern can be deposited using a method that includes moving an assembly supporting the device along a predetermined path while a stationary dispenser assembly deposits the composition onto the device. In other embodiments, both the assembly supporting the device and the dispenser assembly can move to form the preselected pattern on the device.

The preselected geometrical pattern of the coating composition may be applied as a continuous stream that is either in a substantially straight line or a line that has a curved or angular pattern. The preselected geometrical pattern may also be an intermittent pattern that is in a straight line, a line that curved or angular, includes at least one bead, or is a single bead.

In some embodiments, the application of the coating composition on a device is followed by a redistribution of the composition along the device. This redistribution may be accomplished by using, for example, air pressure, centrifugal force, or a second solvent.

After the coating of the stent 603 abluminal surface, the stent 603 can then have other surfaces coated, for example, the inner surface, using other coating methods such as, for example electrospraying or spray coating. Without masking the outer surface of the stent 603, both electrospraying and spray coating may be used to apply a desired composition onto the outer surface and sidewalls of the stent 603. However, the inner surface would be substantially solely coated with a single composition different from the composition used to coat the outer surface of the stent 603. Accordingly, it will be appreciated by one of ordinary skill in the art that this embodiment enables the coating of the inner surface and the outer surface of the stent 603 with different compositions. For example, the luminal surface could be coated with a composition having a desired agent or combination of agents (e.g., an anticoagulant, such as heparin; and/or a non-fouling agent such as a form of PEG) while the abluminal surface of the stent 603 could be coated with a composition having an agent or combination of agents for local delivery to a blood vessel wall (e.g., an anti-inflammatory drug and/or an antiproliferative).

The controlled-volumes of the present invention can be delivered in a system that incorporates a nozzle in the delivery of the coating compositions or a system that can deliver the coating compositions without a nozzle. FIGS. 7a-7c illustrate an assembly that incorporates a nozzle according to some embodiments of the present invention. In some embodiments, the assembly can be used to represent aperture 618 in FIG. 6. Dispenser assembly 701 can be used for a controlled delivery and deposition of composition 702 on a surface of a device.

As shown in FIG. 7a, dispenser assembly 701 can be a simple device comprising a reservoir 703, which holds composition 702 prior to delivery and nozzle 704 having orifice or aperture 705 through which composition 702 is delivered. In one example, the dispenser assembly 701 can be an ink-jet-type printhead. In another example, the dispenser assembly 701 can be a microinjector capable of injecting small volumes ranging from about 2 nL to about 70 nL (e.g., a NanoLiter 2000 available from World Precision Instruments or a Pneumatic PicoPumps PV830 with Micropipette available from Cell Technology System). These microinjection syringes may be employed in conjunction with a microscope of a suitable design.

Nozzle 704 may be permanently, removably or disposably affixed to reservoir 703 and may be made of any suitable material including, but not limited to, glass, metal, sapphire, and plastics. Particular care should be taken to ensure that a glass nozzle 704 does not make contact with the surface of the device upon deposition of the composition 702 to avoid breakage of the nozzle 704. Particular care should also be taken to ensure that a plastic nozzle 704 is compatible with the composition 702. Nozzle 704 may be of any suitable design including, but not limited to, the designs illustrated by FIGS. 7b and 7c. The nozzle 704 depicted in FIG. 7c may be particularly useful for applications in which lifting of a final droplet 706 of composition 702 is desirable, as the depicted design of nozzle 704 allows the capture of final droplet 706 within orifice 705. In addition, dispenser assembly 701 may include more than one reservoir 703 and nozzle 704 to enable dispensing a plurality of coating compositions.

Orifice 705 of the nozzle 704 can range in diameter from about 0.5 μm to about 150 μm. The particular size of orifice 705 depends on factors such as the constituents of composition 702, the viscosity of composition 702 to be applied, the deposition pattern that is desired, and the type of medical device used. For example, a larger orifice 705 may be utilized for application of the composition 702 to the entire outer surface of the medical device than the orifice 705 for the application of the composition 702 into discrete channels or cavities within the medical device. In some embodiments, the orientation of the central axis of nozzle 704 during application of the coating composition can be 90° to the surface of the device that is being coated. In other embodiments, the orientation of the central axis of nozzle 704 during application of the coating composition can be less than 90° to the surface of the device that is being coated.

Delivery of the composition 702 using dispenser assembly 701 can be achieved either passively or actively. In some embodiments, delivery can be achieved passively through capillary action. Alternatively, and as described above, delivery can also be achieved actively by applying a source of pressure (P) to the composition 702 in reservoir 703 as depicted in FIG. 7a. Continuous pressure is applied if deposition of a continuous stream of the composition 702 is desired. Bursts of pressure can be employed if an intermittent deposition pattern of the composition 702 is desired. Any forms of pressure known and available to one of ordinary skill in the art can be used.

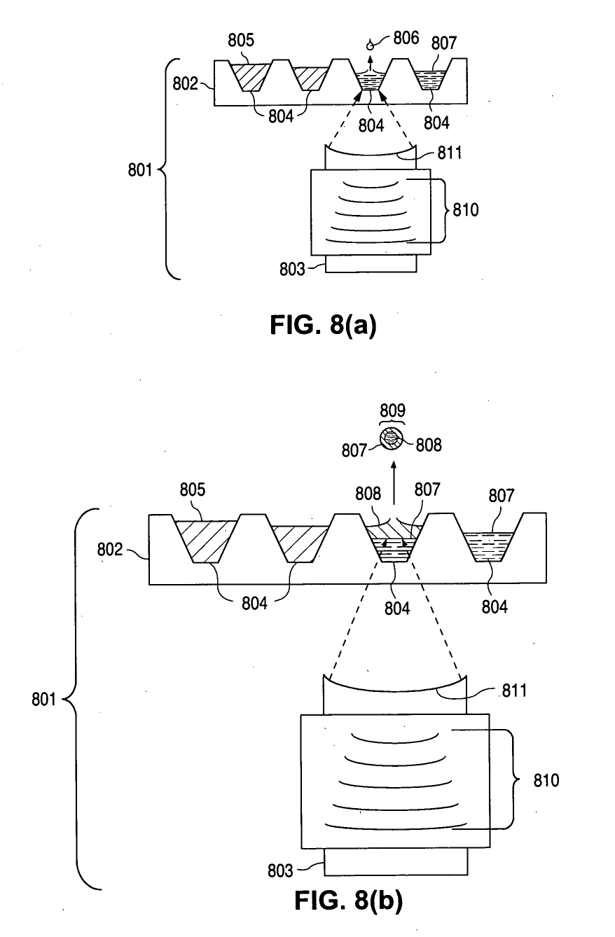

FIGS. 8a and 8b illustrate an ejector assembly that does not require a nozzle, according to some embodiments of the present invention. In some embodiments, the ejector assembly 801 can be used to represent aperture 618 in FIG. 6 for controlled delivery of a coating composition that does not require a nozzle. FIG. 8a illustrates a cross section of the ejector assembly 801 comprising a reservoir housing 802 and a transducer 803. The transducer 803 outputs acoustic energy at a reservoir 804 focused at the surface of the coating composition 805 therein. Each pulse ejects a known amount of the coating composition 805 in a droplet 806 from the reservoir 804 onto a medical device, thereby decreasing the coating composition 805 level in the reservoir 804. Accordingly, after each pulse of acoustic energy, the transducer 803 can be refocused to the new level in the reservoir 804.

In an alternative embodiment, the reservoir 804 can be constantly refilled, thereby keeping the coating composition 805 level the same throughout the coating process. In some embodiments of the invention, the reservoirs 804 can each hold different coating substances. In one example, a first reservoir can hold coating composition 805 while a second reservoir can hold coating composition 807. The transducer 803 can then cause the ejection of different coating substances onto the medical device during a single coating process. Further, since there is no contact between the transducer 803 and reservoirs 804, the chance of cross contamination between reservoirs 804 is minimized or eliminated and there is no possibility of clogging any ejector assembly 801.

In the embodiment shown in FIG. 8b, one or more of the reservoirs 804 may contain two different coating substances: a first substance 807 and a second substance 808, such that the transducer 803 can eject a combined drop 809 from the reservoir 804 by focusing a pulse of acoustic energy 810 at the interface between the two substances. The pulse of acoustic energy 810 is focused by a lens 811. Accordingly, in some embodiments, the medical device can be coated simultaneously with two different coating substance, such as a first substance 807 encapsulating a second substance 808. In some embodiments, the first substance 807 can be a biodegradable polymer selected to control the release of second substance 808, which can be a desired bioactive agent. In other embodiments, the first substance 807 can be a first agent, and the second substance 808 can be a second agent, wherein the agents can be any agent taught herein.

An advantage of the ejector assembly 801 illustrated in FIGS. 8a and 8b is the improved ability to eject controlled-volumes, such as droplets, in a true “drop-on-demand,” or “monodispersed” form. In some embodiments, the controlled-volumes can be delivered in specific locations drop-by-drop. In other embodiments, the controlled-volumes can be delivered in a continuous string using, for example, high frequency acoustic energy.

The controlled-volumes can be delivered in a variety of sizes. In some embodiments, the controlled-volumes can be dispersed in volumes that range from about 1 femtoliter to about 1 microliter, from about 1 femtoliter to about 100 nanoliters, from about 1 femtoliter to about 10 nanoliters, from about 10 femtoliters to about 0.1 nanoliters, from about 10 femtoliters to about 100 picoliters, from about 100 femtoliters to about 10 picoliters, and any range therein. In some embodiments, the controlled-volume is smaller than 10 picoliters to assist in even distribution of monodisperse droplets. An advantage of this broad range of controlled-volumes is that extremely potent agents can be delivered alone in the desired quantities to a desired area on a surface of a medical device. Another advantage of this broad range of controlled-volumes is that multiple agents can be delivered independently, or in combination, in a range of quantities to a range of desired areas and on multiple surfaces of a medical device.

Another advantage of the ejector assembly 801 is that the system can be designed to eject the controlled volumes either upward or downward. FIG. 9 illustrates a method of ejecting the controlled-volumes downward onto the abluminal surface of a stent according to some embodiments of the present invention. The ejector assembly 901 focuses acoustic energy 902 from a transducer 903 with a lens 904. Monodispersed droplets, or controlled-volumes 905, are created at the fluid meniscus 906 created by a coating composition 907 at aperture 908. The desired agents can be positioned within the monodispersed droplets 905 at predetermined regions on a device or within a coating on the device, such as an abluminal surface of a stent 909.

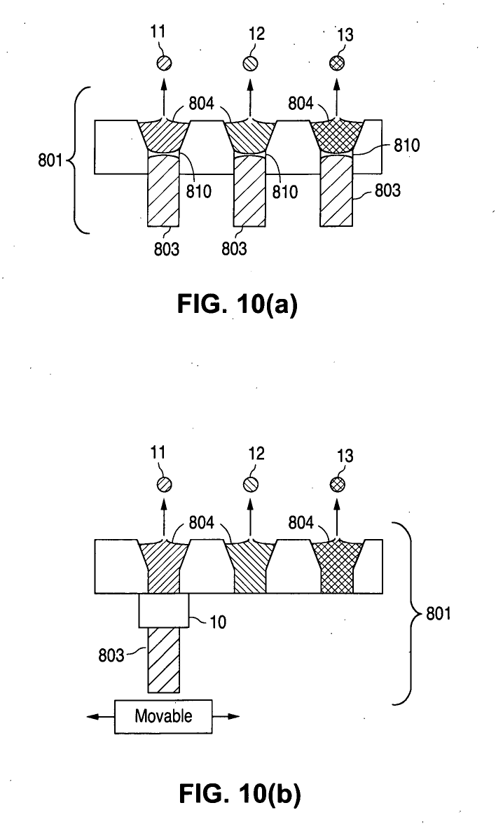

FIGS. 10a and 10b illustrate alternative designs of an acoustic ejector assembly according to some embodiments of the present invention. As shown in FIG. 10, the ejector assembly 801, as first shown in FIGS. 8a and 8b, can be designed such that the transducer 803 and lens 810 are in direct contact with the coating composition 805 or indirectly in contact with the coating composition 805 through a coupling fluid 10. The direct contact design shown in FIG. 10a uses a transducer 803 for each of the reservoirs 804, whereas the indirect contact design shown in FIG. 10b uses a single transducer 803 for a plurality of reservoirs 804. In either embodiment, the ejector assembly 801 has the ability to monodisperse and apply a multitude of agents 11, 12, 13 at predetermined regions on a device or within a coating on the device, such as the abluminal surface of a stent 909 as shown in FIG. 9.

The coating processes taught herein may involve use of a casting solvent. A casting solvent is a liquid medium within which a polymer can be solubilized to form a solution that may be applied as a coating on a substrate. The casting solvent must be selected to avoid adversely affecting an underlying material such as, for example, an underlying primer layer or a bare stent structure. In one example, a material used to form the primer layer is soluble in a highly polar casting solvent but is reasonably insoluble in a low polarity casting solvent. A material is “reasonably insoluble” in a solvent when the material does not solubilize to an extent great enough to significantly affect the performance of the resulting product, meaning that the product can still be used for its intended purpose. In this example, an overlying agent layer that is soluble in a low polarity casting solvent can be applied to the underlying primer layer without disrupting the structure of primer layer.

The casting solvent may be chosen based on several criteria including, for example, its polarity, ability to hydrogen bond, molecular size, volatility, biocompatibility, reactivity and purity. Other physical characteristics of the casting solvent may also be taken into account including the solubility limit of the polymer in the casting solvent, the presence of oxygen and other gases in the casting solvent, the viscosity and vapor pressure of the combined casting solvent and polymer, the ability of the casting solvent to diffuse through an underlying material, and the thermal stability of the casting solvent.

One of skill in the art has access to scientific literature and data regarding the solubility of a wide variety of polymers. Furthermore, one of skill in the art will appreciate that the choice of casting solvent may begin empirically by calculating the Gibb's free energy of dissolution using available thermodynamic data. Such calculations allow for a preliminary selection of potential solvents to test in a laboratory. It is recognized that process conditions can affect the chemical structure of the underlying materials and, thus, affect their solubility in a casting solvent. It is also recognized that the kinetics of dissolution are a factor to consider when selecting a casting solvent, because a slow dissolution of an underlying material, for example, may not affect the performance characteristics of a product where the product is produced relatively quickly.

Exemplary casting solvents for use in the present invention include, but are not limited to, DMAC, DMF, THF, cyclohexanone, xylene, toluene, acetone, i-propanol, methyl ethyl ketone, propylene glycol monomethyl ether, methyl butyl ketone, ethyl acetate, n-butyl acetate, and dioxane. Solvent mixtures can be used as well. Representative examples of the mixtures include, but are not limited to, DMAC and methanol (50:50 w/w); water, i-propanol, and DMAC (10:3:87 w/w); i-propanol and DMAC (80:20, 50:50, or 20:80 w/w); acetone and cyclohexanone (80:20, 50:50, or 20:80 w/w); acetone and xylene (50:50 w/w); acetone, xylene and FLUX REMOVER AMSO (93.7% 3,3-dichloro-1,1,1,2,2-pentafluoropropane and 1,3-dichloro-1,1,2,2,3-pentafluoropropane, and the balance is methanol with trace amounts of nitromethane; Tech Spray, Inc.) (10:40:50 w/w); and 1,1,2-trichloroethane and chloroform (80:20 w/w).

It should be appreciated that a process of forming a medical article or coating can include additional process steps such as, for example, the use of energy such as heat, electromagnetic radiation, electron beam, ion or charged particle beam, neutral-atom beam, and chemical energy. The process of drying can be accelerated by using higher temperatures. In some embodiments, the control of the application of energy includes manual control by the operator. In other embodiments, the control of the application of energy includes a programmable heating control system. In some embodiments, the application of energy can result in a coating composition temperature that ranges from about 35° C. to about 100° C., from about 35° C. to about 80° C., from about 35° C. to about 55° C., or any range therein. In some embodiments, any procedure for drying or curing known to one of skill in the art is within the scope of this invention.

A medical article or coating can also be annealed to enhance the mechanical properties of the composition. Annealing can be used to help reduce part stress and can provide an extra measure of safety in applications such as complex medical devices, where stress-cracking failures can be critical. The annealing can occur at a temperature that ranges from about 30° C. to about 200° C., from about 35° C. to about 190° C., from about 40° C. to about 180° C., from about 45° C. to about 175° C., or any range therein. The annealing time can range from about 1 second to about 60 seconds, from about 1 minute to about 60 minutes, from about 2 minute to about 45 minutes, from about 3 minute to about 30 minutes, from about 5 minute to about 20 minutes, or any range therein. The annealing can also occur by cycling heating with cooling, wherein the total time taken for heating and cooling is the annealing cycle time.

The following examples are provided to further illustrate embodiments of the present invention.

### EXAMPLE 1

A medical article with two layers of coating can be fabricated to comprise everolimus and clobetasol by preparing a first composition and a second composition. The first composition can be an agent layer comprising a matrix of a first biodegradable polymer, e.g. poly(L-lactide), and clobetasol; and, the second composition can be an agent layer comprising a matrix of a second biodegradable polymer, e.g. poly(D,L-lactide), and everolimus.

The first composition can be prepared by mixing the first biodegradable polymer with the everolimus in chloroform to form a first coating composition. The first coating composition can be applied in monodispersed form onto an abluminal surface of a bare 12 mm VISION™ stent (Guidant Corp.) (“example stent”) and dried to form a first coating. The second coating composition can be prepared by mixing the second biodegradable polymer with the everolimus in methyl-ethyl-ketone to form a second coating composition. The second coating composition can be applied in monodispersed form in select areas only on the abluminal surface of the stent. The monodispersed drop size can range from about 1 picoliter to about 10 picoliters.

A topcoat layer can optionally be applied to assist in control of release of the agents. An example coating technique for the topcoat layer comprises applying a poly(hydroxyalkanoate)/ethanol mixture onto the coated abluminal surface of the stent. Bake the coating at about 50° C. for about 1 hour after the final pass to form a dry agent layer.

### EXAMPLE 2

A medical article with three layers of coating can be fabricated to comprise everolimus and tacrolimus by preparing a first composition, a second composition and a third composition. The first composition can be a primer layer of a mixture of a poly(hydroxyalkanoate) and tacrolimus. The second composition can be a pure agent layer of everolimus, and the third composition can be a topcoat layer of a poly(hydroxyalkanoate).

The first composition can be prepared by mixing about 2% (w/w) of the poly(hydroxyalkanoate) in absolute ethanol with an adequate amount of tacrolimus and can be applied onto the surface of the example stent using the acoustic ejector assembly technique to form a dry primer layer. The dry primer layer can contain about 100 μg of the poly(hydroxyalkanoate) combined with the adequate amount of tacrolimus. The second composition can be prepared by mixing about 2% (w/w) everolimus in absolute ethanol and applying the mixture to the primer layer using acoustic ejector assembly technique to form a pure agent layer comprising controlled volumes of everolimus. The third composition can be prepared by mixing about 2% (w/w) of the poly(hydroxyalkanoate) in absolute ethanol and applying the mixture using the example coating technique of Example 1 to form a topcoat layer comprising the poly(hydroxyalkanoate).

While particular embodiments of the present invention have been shown and described, those skilled in the art will note that variations and modifications can be made to the present invention without departing from the spirit and scope of the teachings. A multitude of coating apparatuses, polymers, agents and methods of forming controlled-volumes for the production of medical devices have been taught herein. One of skill in the art is to appreciate that such teachings are provided by way of example only and are not intended to limit the scope of the invention.

We claim:

1. A medical device comprising a combination of agents, wherein an agent within the combination of agents is positioned within a controlled volume at one or more predetermined regions on a medical device, within the medical device, within a coating on the medical device, or a combination thereof.

2. The medical device of claim 1, wherein the combination of agents comprises a bioactive agent, a biobeneficial agent, a diagnostic agent, a plasticizing agent or a combination thereof.

3. The medical device of claim 1, wherein the agent comprises a component selected from a group consisting of poly(alkylene glycols), phosphorylcholine, poly(N-vinyl pyrrolidone), poly(ethylene oxide), poly(acrylamide methyl propane sulfonic acid), poly(styrene sulfonate), polysaccharides, poly(ester amides), peptides, non-thrombotics, antimicrobials, and any derivatives, analogs, homologues, congeners, salts, copolymers and combinations thereof.

4. The medical device of claim 3, wherein the poly(alkylene glycol) comprises a component selected from a group consisting of poly(ethylene glycol), poly(propylene glycol), and any derivatives, analogs, homologues, congeners, salts, copolymers and combinations thereof.

5. The medical device of claim 3, wherein the polysaccharide comprises a component selected from a group consisting of carboxymethylcellulose, sulfonated dextran, sulfated dextran, dermatan sulfate, chondroitin sulfate, hyaluronic acid, heparin, hirudin, and any derivatives, analogs, homologues, congeners, salts, copolymers and combinations thereof.

6. The medical device of claim 3, wherein the peptide comprises a component selected from a group consisting of elastin, silk-elastin, collagen, atrial natriuretic peptide (ANP), Arg-Gly-Asp (RGD); and any derivatives, analogs, homologues, congeners, salts, copolymers and combinations thereof.

7. The medical device of claim 1, wherein the agent comprises a component selected from a group consisting of a free radical scavenger, a nitric oxide donor, rapamycin, methyl rapamycin, everolimus, 42-Epi-(tetrazoylyl)rapamycin (ABT-578), tacrolimus, paclitaxel, docetaxel, estradiol, clobetasol, idoxifen, tazarotene and any prodrugs, metabolites, analogs, homologues, congeners, and any derivatives, salts and combinations thereof.

8. The medical device of claim 7, wherein the free radical scavenger comprises a component selected from a group consisting of 2,2′,6,6′-tetramethyl-1-piperinyloxy, free radical; 4-amino-2,2′,6,6′-tetramethyl-1-piperinyloxy, free radical; 4-hydroxy-2,2′,6,6′-tetramethyl-piperidene-1-oxy, free radical; 2,2′,3,4,5,5′-hexamethyl-3-imidazolinium-1-yloxy methyl sulfate, free radical; 16-doxyl-stearic acid, free radical; superoxide dismutase mimic; and, any analogs, homologues, congeners, derivatives, salts and combinations thereof.

9. The medical device of claim 7, wherein the nitric oxide donor comprises a component selected from the group consisting of S-nitrosothiols, nitrites, N-oxo-N-nitrosamines, substrates of nitric oxide synthase, diazenium diolates and any analogs, homologues, congeners, derivatives, salts and combinations thereof.

10. The medical device of claim 1, wherein the combination of agents comprises everolimus, clobetasol, tacrolimus, rapamycin, ABT-578, or any combination thereof.

11. The medical device of claim 1, wherein a polymer is combined with an agent within and/or encapsulating the controlled volume comprising the agent.

12. The medical device of claim 1, wherein the predetermined regions comprise an abluminal surface of the medical device.

13. The medical device of claim 1, wherein the coating comprises a plurality of layers, and the predetermined regions comprise a location within the plurality of layers.

14. The medical device of claim 1, wherein the controlled volume ranges from about 1 femtoliter to about 100 nanoliters.

15. The medical device of claim 1, comprising an agent that releases from the coating and/or the device at a predetermined rate.

16. The medical device of claim 1, wherein the medical device comprises a stent.

17. A coating for a medical device comprising a combination of agents, wherein an agent is positioned within a controlled volume at one or more predetermined regions on the device, within the device, within a coating on the device, or a combination thereof.

19. The coating of claim 17, wherein the combination of agents comprises a bioactive agent, a biobeneficial agent, a diagnostic agent, a plasticizing agent or a combination thereof.

20. The coating of claim 17, wherein the combination of agents comprises a component selected from a group consisting of poly(alkylene glycols), phosphorylcholine, poly(N-vinyl pyrrolidone), poly(ethylene oxide), poly(acrylamide methyl propane sulfonic acid), poly(styrene sulfonate), polysaccharides, poly(ester amides), peptides, non-thrombotics, antimicrobials, and any derivatives, analogs, homologues, congeners, salts, copolymers and combinations thereof.

21. The coating of claim 20, wherein the poly(alkylene glycol) comprises a component selected from a group consisting of poly(ethylene glycol), poly(propylene glycol), and any derivatives, analogs, homologues, congeners, salts, copolymers and combinations thereof.

22. The coating of claim 20, wherein the polysaccharide comprises a component selected from a group consisting of carboxymethylcellulose, sulfonated dextran, sulfated dextran, dermatan sulfate, chondroitin sulfate, hyaluronic acid, heparin, hirudin, and any derivatives, analogs, homologues, congeners, salts, copolymers and combinations thereof.

23. The coating of claim 20, wherein the peptide comprises a component selected from a group consisting of elastin, silk-elastin, collagen, atrial natriuretic peptide (ANP), Arg-Gly-Asp (RGD); and any derivatives, analogs, homologues, congeners, salts, copolymers and combinations thereof.

24. The coating of claim 17, wherein the agent comprises a component selected from a group consisting of a free radical scavenger, a nitric oxide donor, rapamycin, methyl rapamycin, everolimus, 42-Epi-(tetrazoylyl)rapamycin (ABT-578), tacrolimus, paclitaxel, docetaxel, estradiol, clobetasol, idoxifen, tazarotene and any prodrugs, metabolites, analogs, homologues, congeners, and any derivatives, salts and combinations thereof.

25. The coating of claim 24, wherein the free radical scavenger comprises a component selected from a group consisting of 2,2′,6,6′-tetramethyl-1-piperinyloxy, free radical; 4-amino-2,2′,6,6′-tetramethyl-1-piperinyloxy, free radical; 4-hydroxy-2,2′,6,6′-tetramethyl-piperidene-1-oxy, free radical; 2,2′,3,4,5,5′-hexamethyl-3-imidazolinium-1-yloxy methyl sulfate, free radical; 16-doxyl-stearic acid, free radical; superoxide dismutase mimic; and, any analogs, homologues, congeners, derivatives, salts and combinations thereof.

26. The coating of claim 24, wherein the nitric oxide donor comprises a component selected from the group consisting of S-nitrosothiols, nitrites, N-oxo-N-nitrosamines, substrates of nitric oxide synthase, diazenium diolates and any analogs, homologues, congeners, derivatives, salts and combinations thereof.

27. The coating of claim 17, wherein the combination of agents comprises everolimus, clobetasol, tacrolimus, rapamycin, ABT-578, or any combination thereof.

28. The coating of claim 17, wherein a polymer is combined with an agent within and/or encapsulating the controlled volume comprising the agent.

29. The coating of claim 17, wherein the predetermined regions comprise an abluminal surface of the medical device.

30. The coating of claim 17, wherein the coating comprises a plurality of layers, and the predetermined regions comprises a location within the plurality of layers.

31. The coating of claim 17, wherein the controlled volume ranges from about 1 femtoliter to about 100 nanoliters.

32. The coating of claim 17, comprising an agent that releases from the coating at a predetermined rate.

33. A method of coating a medical device comprising:
selecting a combination of agents; and
applying an agent from the combination of agents within one or more controlled volumes at one or more predetermined regions on a medical device, within the device, within a coating for the device, or a combination thereof, such that the coating comprises the one or more controlled volumes.

34. The method of claim 33, wherein the combination of agents comprises a bioactive agent, a biobeneficial agent, a diagnostic agent, a plasticizing agent or a combination thereof.

35. The method of claim 33, wherein the combination of agents comprises a component selected from a group consisting of poly(alkylene glycols), phosphorylcholine, poly(N-vinyl pyrrolidone), poly(ethylene oxide), poly(acrylamide methyl propane sulfonic acid), poly(styrene sulfonate), polysaccharides, poly(ester amides), peptides, non-thrombotics, antimicrobials, and any derivatives, analogs, homologues, congeners, salts, copolymers and combinations thereof.

36. The method of claim 35, wherein the poly(alkylene glycol) comprises a component selected from a group consisting of poly(ethylene glycol), poly(propylene glycol), and any derivatives, analogs, homologues, congeners, salts, copolymers and combinations thereof.

37. The method of claim 35, wherein the polysaccharide comprises a component selected from a group consisting of carboxymethylcellulose, sulfonated dextran, sulfated dextran, dermatan sulfate, chondroitin sulfate, hyaluronic acid, heparin, hirudin, and any derivatives, analogs, homologues, congeners, salts, copolymers and combinations thereof.

38. The method of claim 35, wherein the peptide comprises a component selected from a group consisting of elastin, silk-elastin, collagen, atrial natriuretic peptide (ANP), Arg-Gly-Asp (RGD); and any derivatives, analogs, homologues, congeners, salts, copolymers and combinations thereof.

39. The method of claim 33, wherein the combination of agents comprises a component selected from a group consisting of a free radical scavenger, a nitric oxide donor, rapamycin, methyl rapamycin, everolimus, 42-Epi-(tetrazoylyl)rapamycin (ABT-578), tacrolimus, paclitaxel, docetaxel, estradiol, clobetasol, idoxifen, tazarotene and any prodrugs, metabolites, analogs, homologues, congeners, and any derivatives, salts and combinations thereof.

40. The method of claim 39, wherein the free radical scavenger comprises a component selected from a group consisting of 2,2′,6,6′-tetramethyl-1-piperinyloxy, free radical; 4-amino-2,2′,6,6′-tetramethyl-1-piperinyloxy, free radical; 4-hydroxy-2,2′,6,6′-tetramethyl-piperidene-1-oxy, free radical; 2,2′,3,4,5,5′-hexamethyl-3-imidazolinium-1-yloxy methyl sulfate, free radical; 16-doxyl-stearic acid, free radical; superoxide dismutase mimic; and, any analogs, homologues, congeners, derivatives, salts and combinations thereof.

41. The method of claim 38, wherein the nitric oxide donor comprises a component selected from the group consisting of S-nitrosothiols, nitrites, N-oxo-N-nitrosamines, substrates of nitric oxide synthase, diazenium diolates and any analogs, homologues, congeners, derivatives, salts and combinations thereof.

42. The method of claim 33, wherein the combination of agents comprises everolimus, clobetasol, tacrolimus, rapamycin, ABT-578, or any combination thereof.

43. The method of claim 33, wherein a polymer is combined with an agent from the combination of agents within the one or more controlled volumes comprising the agent.

44. The method of claim 33, wherein the controlled volumes range from about 1 femtoliter to about 100 nanoliters.

45. The method of claim 33, wherein the medical device comprises a stent.

46. The method of claim 33, wherein the predetermined regions comprise the abluminal surface of the stent.

47. The method of claim 33, wherein the coating comprises a plurality of layers, and the predetermined regions comprise a location throughout the plurality of layers.

48. The method of claim 33, wherein the selecting further comprises designing the combination of agents such that an agent releases from the coating at a predetermined rate.

49. The method of claim 33, wherein the applying comprises forming the controlled volumes through the use of acoustic energy.

50. A coating for a medical device comprising a combination of agents, wherein the coating is formed using a process comprising:
selecting a combination of agents, wherein the combination of agents comprises everolimus, clobetasol, tacrolimus, rapamycin, ABT-578, or any combination thereof; and
applying an agent from the combination of agents within one or more controlled volumes at one or more predetermined regions on a medical device, within the device, within a coating for the device, or a combination thereof, such that the coating comprises the one or more controlled volumes; wherein, the applying comprises forming the controlled volumes through a method comprising the use of acoustic energy.
